SEQUENCE LISTING (12 sequences)

SEQ ID NO: 1 - DNA, 22 bp, Artificial
  SYNTHETIC OLIGONUCLEOTIDE PRIMER
gtcaacatagcagaataggcat

SEQ ID NO: 2 - DNA, 21 bp, Artificial
  SYNTHETIC OLIGONUCLEOTIDE PRIMER
gtacccatccggatatagttc

SEQ ID NO: 3 - protein, 5 aa, unknown
  ENDOGENOUS OPIOID PEPTIDE MET-ENKEPHALIN
YGGFM

SEQ ID NO: 4 - protein, 5 aa, unknown
  ENDOGENOUS OPIOID PEPTIDE LEU-ENKEPHALIN
YGGFL

SEQ ID NO: 5 - protein, 8 aa, unknown
  ENDOGENOUS OPIOID PEPTIDE OCTAPEPTIDE
YGGFMRGL

SEQ ID NO: 6 - protein, 7 aa, unknown
  ENDOGENOUS OPIOID PEPTIDE HEPTAPEPTIDE
YGGFMRF

SEQ ID NO: 7 - protein, 8 aa, unknown
  ENDOGENOUS OPIOID PEPTIDE DYNORPHIN 1-8
YGGFLRRI

SEQ ID NO: 8 - protein, 17 aa, unknown
  ENDOGENOUS OPIOID PEPTIDE DYNORPHIN 1-17
YGGFLRRIRPKLKWDNQ

SEQ ID NO: 9 - protein, 10 aa, unknown
  ENDOGENOUS OPIOID PEPTIDE A-NEOENDORPHIN
YGGFLRKYPK

SEQ ID NO: 10 - protein, 9 aa, unknown
  ENDOGENOUS OPIOID PEPTIDE B-NEOENDORPHIN
YGGFLRKYP

SEQ ID NO: 11 - protein, 4 aa, unknown
  ENDOGENOUS OPIOID PEPTIDE ENDOMORPHIN 1
YPWF

SEQ ID NO: 12 - protein, 4 aa, unknown
  ENDOGENOUS OPIOID PEPTIDE ENDOMORPHIN 2
YPFF

(19) United States
(12) Patent Application Publication
(10) Pub. No.: US 2006/0019241 A1
(43) Pub. Date: Jan. 26, 2006

(54) Na+ and CI-coupled transport system for endogenous opioid peptides

(76) Inventors: Vadivel Ganapathy, Martinez, GA (US); Seiji Miyauchi, Kita-Ku (JP)

Correspondence Address: MUETING, RAASCH & GEBHARDT, P.A., P.O. BOX 581415, MINNEAPOLIS, MN, 55458, US

(21) Appl. No.: 11/110,619

(22) Filed: Apr. 20, 2005

Related U.S. Application Data
(60) Provisional application No. 60/563,768, filed on Apr. 20, 2004.

(51) Int. Cl. C12Q 1/70; G01N 33/53; C07K 14/72; G01N 33/567

(52) U.S. Cl. 435/5; 435/7.2; 530/350

(57) ABSTRACT

The present invention provides the identification and characterization of an endogenous opioid peptide transporter, and uses thereof.

This application claims the benefit of U.S. Provisional Application Ser. No. 60/563,768, filed Apr. 20, 2004, which is incorporated by reference herein.

### GOVERNMENT FUNDING

The present invention was made with government support under Grant No. HD-44404, awarded by the National Institutes of Health. The Government may have certain rights in this invention.

### BACKGROUND

Opioidergic neurotransmission plays a critical role in a variety of biological processes, including analgesia, constipation, respiration, euphoria, sedation, and meiosis (Akil et al, (1984) Annu. Rev. Neurosci. 7: 223-255; De Luca et al., (1996) Pharmacol. Ther. 69: 103-115; Okada et al., (2002) Vitam. Horm. 65: 257-279; and Bodnar and Hadjimarkou, (2003) Peptides 24: 1241-1302). Three distinct types of opiate receptors have been identified at the molecular level: μ (mu), δ (delta), and κ (kappa) (Massotte and Kieffer, (1998) Essays Biochem. 33: 65-77; and Waldhoer et al., (2004) Annu. Rev. Biochem. 73: 953-990). The action of opiates in inducing analgesia and constipation has tremendous therapeutic applications as evidenced by the current use of various opiate agonists as potent analgesics and anti-diarrheal agents. Available evidence indicates that while the analgesic effects of opiates are mediated by μ and δ receptors, the anti-diarrheal effect may involve all three receptor subtypes (Bauer, A. J., Sarr, M. G. and Szurszewski, J. H. (1991) Gastroenterology 101: 970-976; and Holzer, P. (2004) Neurosci. Lett. 361: 192-195).

The discovery of receptors for exogenous opiates such as morphine has led to the identification of various endogenous opiates which function as physiological ligands for these receptors. There are four classes of endogenous opioid peptides: enkephalins, endomorphins, dynorphins, and endorphins (Akil et al, (1984) Annu. Rev. Neurosci. 7: 223-255; De Luca et al., (1996) Pharmacol. Ther. 69: 103-115; Okada et al., (2002) Vitam. Horm. 65: 257-279; and Bodnar and Hadjimarkou, (2003) Peptides 24: 1241-1302). These peptides are produced in vivo from different precursor proteins that are found primarily in the brain and gastrointestinal tract. Thus, the brain and gastrointestinal tract represent the primary targets for these opioid peptides. These peptides produce their biological effects in mammalian cells by interacting with different subtypes of opiate receptors located on the plasma membrane and in the nucleus. As shown in FIG. 19, there is significant specificity in the interaction of various opioid peptides with the three known different opiate receptors.

Activation of opioidergic neurotransmission facilitates analgesia and constipation, providing a basis for the therapeutic potential of opiate agonists as analgesics and anti-diarrheal agents and in the management of diarrhea-predominant irritable bowel syndrome. As in any neurotransmission process, the magnitude of opioidergic neurotransmission depends on the concentration of opioid peptides in the synaptic cleft. This in turn depends on the cellular processes involved in the clearance of opioid peptides from the synapse. While the mechanisms of clearance from the synapse have been well studied in the case of various neurotransmitters such as acetylcholine (acetylcholineesterase) and monoamines (monoamine transporters), very little is known on the molecular processes responsible for the clearance of opioid peptides from the synapse.

### SUMMARY OF THE INVENTION

The present invention includes a method of identifying an agent that modulates the transmembrane transport of an endogenous opioid peptide by an endogenous opioid peptide transport system, the method including contacting a cell expressing an endogenous opioid peptide transport system with an agent; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe; and determining the transmembrane transport of the endogenous opioid peptide by the endogenous opioid peptide transport system; wherein a modulation in the transmembrane transport of the endogenous opioid peptide when the cell is contacted with the agent indicates the agent modulates the transmembrane transport of an opioid peptide by an endogenous opioid peptide transport system.

In some aspects of the method, the endogenous opioid peptide transport system exhibits upregulation of the transport of endogenous opioid peptide by the Tat protein encoded by the human immunodeficiency virus type I.

In some aspects of the method, the endogenous opioid peptide transport system exhibits transport of an endogenous opioid peptide that is coupled to a sodium gradient.

In some aspects of the method, the endogenous opioid peptide transport system exhibits transport of an endogenous opioid peptide that is coupled to a chloride gradient.

In some aspects of the method, the endogenous opioid peptide transport system exhibits inhibition of the transport of endogenous opioid peptide by L-lysine.

In some aspects of the method, the endogenous opioid peptide transport system exhibits stimulation of the transport of endogenous opioid peptide by the tripeptides Gly-Gly-Ile and/or Gly-Gly-Phe.

In some aspects of the method, the endogenous opioid peptide transport system exhibits upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and stimulation of the transport of endogenous opioid peptide by the tripeptides Gly-Gly-Ile and/or Gly-Gly-Phe.

The present invention also includes a method of modulating the activity of an opioid the method including administering an agent that modulates the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects of the method, the agent may be Gly-Gly-Ile, Gly-Gly-Phe, Gly-Gly-Gly, Try-Gly-Gly, Glu-Gly-Phe, L-lysine, L-valine, D-alanine, D-tyrosine, L-arginine, analogs or structural derivatives of each, or a combination thereof. In some aspects of the method, modulation may be an increase in the transport of an endogenous opioid peptide and the agent may be, for example, the tripeptide Gly-Gly-Ile, the tripeptide Gly-Gly-Phe, the tripeptide Gly-Gly-Gly, the tripeptide Try-Gly-Gly, the tripeptide Glu-Gly-Phe, an analog or structural derivative of each tripeptide, or a combination thereof. In some aspects of the method, modulation may be an inhibition of the transport of an endogenous opioid peptide, and the agent may be, for example, L-lysine, L-valine, D-alanine, D-tyrosine, L-arginine, an analog or structural derivative of each, or a combination thereof. In one embodiment, the agent is L-lysine or an analog or structural derivative thereof.

Also included in the present invention is a method of treating pain, the method including administering an effective amount of an agent that inhibits the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine; and/or stimulation of the transport of endogenous opioid peptide by the tripeptides Gly-Gly-Ile and/or Gly-Gly-Phe. In some aspects of the method the agent may be, for example, L-lysine, L-valine, D-alanine, D-tyrosine, L-arginine, an analog or structural derivative of each, or a combination thereof. In one embodiment, the agent may be L-lysine or an analog or structural derivative thereof.

Also included in the present invention is a method of reducing the amount of narcotic needed for effective pain management, the method including administering an effective amount of an agent that inhibits the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects of the method, the likelihood of the development of addiction is reduced. In some aspects of the method, the agent may be, for example, L-lysine, L-valine, D-alanine, D-tyrosine, L-arginine, an analog or structural derivative of each, or a combination thereof. In one embodiment, the agent is L-lysine or an analog or structural derivative thereof.

The present invention also includes a method of decreasing the motility of the intestine, the method including administering an effective amount of an agent that inhibits the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects of the method, the agent may be, for example, L-lysine, L-valine, D-alanine, D-tyrosine, L-arginine, an analog or structural derivative or each, or a combination thereof. In one embodiment, the agent is L-lysine or an analog or structural derivative thereof.

The present invention includes a method of treating irritable bowel syndrome (IBS) with diarrhea, the method including administering an effective amount of an agent that inhibits the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type 1, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects of the method, the agent may be, for example, L-lysine, L-valine, D-alanine, D-tyrosine, L-arginine, an analog or structural derivative of each, or a combination thereof. In one embodiment, the agent is L-lysine or an analog or structural derivative thereof.

The present invention includes a method of treating pain in a person infected with human immunodeficiency virus (HIV), the method including administering an effective amount of an agent that inhibits the transmembrane transport of an endogenous opioid peptide by the endogenous opioid peptide transport system; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects of the method, the need to administer a pain-relieving narcotic is reduced. In some aspects of the invention, the person infected with HIV has a history of drug abuse. In some aspects of the present invention, the person with HIV has opiate-resistant pain. In some aspects of the agent may be, for example, L-lysine, L-valine, D-alanine, D-tyrosine, L-arginine, an analog or structural of each, or a combination thereof. In one embodiment, the agent is L-lysine or an analog or structural derivative thereof.

Also included in the present invention are isolated polynucleotides hybridizing under standard hybridization conditions to a polynucleotide sequence that encodes an endogenous opioid peptide transport system polypeptide; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe.

The isolated polynucleotide of the present invention may encode an endogenous opioid peptide transport system polypeptide; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe. In some aspects, the invention includes plasmids and host cells including the isolated polynucleotide. The plasmid may be, for example, an expression vector. The host cell may demonstrate transient expression of a polynucleotide encoding an endogenous opioid peptide transport system polypeptide. The host cell may demonstrate stable expression of a polynucleotide encoding an endogenous opioid peptide transport system polypeptide.

The present invention also includes an isolated polypeptide having at least 70% sequence identity with an endogenous opioid peptide transport system polypeptide; wherein the endogenous opioid peptide transport system exhibits one or more of the following functional activities: upregulation of the transport of endogenous opioid peptide by the Tat protein encoded by the human immunodeficiency virus type I, transport of an endogenous opioid peptide that is coupled to a sodium gradient, transport of an endogenous opioid peptide that is coupled to a chloride gradient, inhibition of the transport of endogenous opioid peptide by L-lysine, and/or stimulation of the transport of endogenous opioid peptide by the tripeptides Gly-Gly-Ile and/or Gly-Gly-Phe.

The present invention includes a transgenic non-human animal transgenic for a polynucleotide hybridizing under standard hybridization conditions to a polynucleotide sequence that encodes an endogenous opioid peptide transport system polypeptide; wherein the endogenous opioid peptide transport system exhibits one or more of the following functional activities: upregulation of the transport of endogenous opioid peptide by the Tat protein encoded by the human immunodeficiency virus type I; transport of an endogenous opioid peptide is coupled to a sodium gradient; transport of an endogenous opioid peptide is coupled to a chloride gradient; inhibition of the transport of endogenous opioid peptide by L-lysine; and/or stimulation of the transport of endogenous opioid peptide by the tripeptides Gly-Gly-Ile and/or Gly-Gly-Phe.

The present invention includes a non-human animal having a knockout mutation in one or more alleles encoding a polypeptide having at least 70% sequence identity with an endogenous opioid peptide transport system polypeptide; wherein the endogenous opioid peptide transport system exhibits one or more of the following functional activities: upregulation of the transport of endogenous opioid peptide by the Tat protein encoded by the human immunodeficiency virus type I; transport of an endogenous opioid peptide is coupled to a sodium gradient; transport of an endogenous opioid peptide is coupled to a chloride gradient; inhibition of the transport of endogenous opioid peptide by L-lysine; and/or stimulation of the transport of endogenous opioid peptide by the tripeptides Gly-Gly-Ile and/or Gly-Gly-Phe.

Unless otherwise specified, “a,” “an,” “the,” and “at least one” are used interchangeably and mean one or more than one.

### BRIEF DESCRIPTION OF THE DRAWINGS

FIGS. 1A-1B present the time course and ion-dependence of deltorphin uptake in pcDNA-ARPE-19 and Tat-ARPE-19 cells. FIG. 1A shows the uptake of [³H]deltorphin II (50 nM) measured in pcDNA-ARPE-19 and Tat-ARPE-19 cells for different periods in the presence of NaCl. FIG. 1B shows the uptake of [³H]deltorphin II (50 nM) measured in pcDNA-ARPE-19 and Tat-ARPE-19 cells for 30 minutes in the presence of Na⁺ and Cl⁻ (NaCl), in the absence of Na⁺ but in the presence of Cl⁻ (NMDG chloride), or in the absence of Cl⁻ but in the presence of Na⁺ (Na gluconate).

FIGS. 2A-2C present saturation kinetics and Na⁺- and Cl⁻-activation kinetics of deltorphin uptake in Tat-ARPE-19 cells. FIG. 2A shows the uptake of deltorphin II measured in the presence of NaCl for 30 minutes over a deltorphin II concentration range of 10-1000 μM. Inset: Eadie-Hofstee plot. FIG. 2B shows the uptake of [³H]deltorphin II (50 nM) measured for 30 minutes in the presence of varying concentrations of Na⁺ (2.5-140 mM), with Cl⁻ concentration kept constant at 140 mM. Inset: Hill plot. FIG. 2C shows the uptake of [³H]deltorphin II (50 nM) measured for 30 minutes in the presence of various concentrations of Cl⁻ (10-140 mM), with Na⁺ concentration kept constant at 140 mM. Inset: Hill plot.

FIGS. 3A-3B show the time course and ion-dependence of deltorphin II uptake in SK—N—SH cells. FIG. 3A represents the uptake of [³H]deltorphin II (25 nM) measured in SK—N—SH cells in the presence of NaCl for varying time periods. FIG. 3B represents the uptake of [³H]deltorphin II (25 nM) measured in SK—N—SH cells under various ionic conditions with a 30 minute incubation.

FIG. 4 demonstrates substrate selectivity of the deltorphin II uptake system in SK—N—SH cells. The substrate selectivity of the uptake process was studied by assessing the effect of various enkephalins and dynorphins on the uptake of [³H]deltorphin II (25 nM) in SK—N—SH cells at enkephalins and dynorphins concentrations of 1 mM.

FIGS. 5A-5B show the relative affinities of various enkephalins and dynorphins for the transport system in SK—N—SH cells. FIG. 5A represents the affinities of Met-enkephalin, Leu-enkephalin, and deltorphin II for the transport system as determined by assessing the concentration-dependent inhibition of the uptake of [³H]deltorphin II (25 nM) in SK—N—SH cells. FIG. 5B represents the affinities of Dynorphin A1-6, Dynorphin A1-7, and Dynorphin A1-13 for the transport system as determined by assessing the concentration-dependent inhibition of the uptake of [³H]deltorphin II (25 nM) in SK—N—SH cells.

FIG. 6 demonstrates the influence of various dipeptides on the enkephalin/endorphin transport system. The influence of various dipeptides on the enkephalin/endorphin transport system in SK—N—SH cells was studied by assessing their effects on the uptake of [³H]deltorphin II (25 nM) in the presence of NaCl for 30 minutes at a dipeptide concentration of 1 mM.

FIG. 7 demonstrates the influence of various tripeptides on the enkephalin/endorphin transport system. The influence of various tripeptides on the enkephalin/endorphin transport system in SK—N—SH cells was studied by assessing their effects on the uptake of [³H]deltorphin II (25 nM) in the presence of NaCl for 30 minutes at a tripeptide concentration of 1 mM.

FIG. 8 demonstrates dose-response relationships for the stimulatory effect of Gly-Gly-Ile and Gly-Gly-Phe. The uptake of [³H]deltorphin II (25 nM) in SK—N—SH cells was measured in the presence of NaCl with a 30 minute incubation in the presence of varying concentrations of the two tripeptides.

FIG. 9 demonstrates the ion-dependence of the enkephalin/endorphin transport system in the absence and presence of the stimulatory modifier Gly-Gly-Ile. The uptake of [³H]deltorphin II (25 nM) in SK—N—SH cells was measured in the absence or presence of 1 mM Gly-Gly-Ile. The uptake buffer contained NaCl (presence of both Na⁺and Cl⁻), NMDG chloride (absence of Na⁺ but presence of Cl⁻), or Na gluconate (presence of Na⁺ but absence of Cl⁻).

FIG. 10 demonstrates the substrate selectivity of the enkephalin/endorphin transport system in the absence and presence of a stimulatory modifier. The substrate selectivity of the transport system was studied in the absence or presence of 1 mM Gly-Gly-Ile (GGI) by assessing the influence of various enkephalins, endorphins, and amino acids on the uptake of [³H]deltorphin II (25 nM) in SK—N—SH cells.

FIGS. 11A-11B show the influence of Gly-Gly-Ile (GGI) on the kinetic parameters of enkephalin/endorphin transport system. The kinetics of deltorphin II uptake in SK—N—SH cells in the absence or presence of 1 mM Gly-Gly-Ile was determined. FIG. 11A represents deltorphin II concentration versus deltorphin II uptake. FIG. 11B represents deltorphin II uptake/deltorphin II concentration (V/s) versus deltorphin II uptake (V).

FIG. 12 demonstrates the influence of Gly-Gly-Ile (GGI) on the Na⁺-activation kinetics of the enkephalin/endorphin transport system. The uptake of [³H]deltorphin II (25 nM) was measured in the absence or presence of Gly-Gly-Ile (1 mM) with varying concentrations of Na⁺ and with a fixed concentration of Cl⁻.

FIG. 13 demonstrates the influence of Gly-Gly-Ile (GGI) on the Cl⁻-activation kinetics of the enkephalin/endorphin transport system. The uptake of [³H]deltorphin II (25 nM) was measured in the absence or presence of Gly-Gly-Ile (1 mM) with varying concentrations of Cl⁻ and with a fixed concentration of Na⁺.

FIG. 14 demonstrates the influence of various amino acids on the enkephalin/endorphin transport system in SK—N—SH cells. The uptake of [³H]deltorphin II (25 nM) was measured in SK—N—SH cells in the presence of NaCl for 30 minutes in the absence or presence of various amino acids (1 mM).

FIG. 15 demonstrates the dose-response relationship for the inhibition of the enkephalin/endorphin transport system by L-Lysine and its methyl and ethyl esters. The uptake of [³H]deltorphin II (25 nM) was measured in SK—N—SH cells in the presence of NaCl for 30 minutes in the absence or presence of increasing concentrations of L-Lysine (Lys) or its esters (Lys-ME and Lys-EE).

FIGS. 16A-16B demonstrate the influence of L-Lysine (Lys) on the kinetics of the enkephalin/endorphin transport system. The kinetics of the enkephalin/endorphin transport system was studied in the absence or presence of 250 μM L-Lysine by using deltorphin II as the substrate for the transport system. FIG. 16A represents Deltorphin It concentration versus deltorphin It uptake. FIG. 16B represents Deltorphin II uptake/deltorphin II concentration (V/S) versus deltorphin II uptake (V).

FIG. 17 shows that the uptake of deltorphin II (25 nM) in primary neuronal cultures from rat brain striatum is stimulated 2-fold in the presence of Na⁺.

FIG. 18 shows the induction of deltorphin uptake in X. laevis oocytes injected with mRNA form the rat Muller cell line rMC-1. Uptake is completely inhibited by dynorphin B1-9 (DynB1-9) and only slightly inhibited by estrone-3-sulfate (Estrone SO₄).

FIG. 19 shows the specificity of the interactions of various endogenous opioid peptides with the three known opiate receptors.

### DETAILED DESCRIPTION OF PREFERRED EMBODIMENTS OF THE INVENTION

The present invention provides the identification of a novel transport system for the transmembrane transport of opioid peptides in mammalian cells that is distinct from any of the previously identified transport systems for opioid peptides. This opioid transport system transports a variety of opioid peptides, including endogenous opioid peptides and analogs thereof. The “opioid transport system” of the present invention may also be referred to herein as an “opioid peptide transport system,” an “endogenous opioid peptide transport system,” an “opioid peptide transporter” or an “endogenous opioid peptide transporter.”

As used herein, an “endogenous opioid peptide” includes naturally occurring or synthetic peptides that bind to or otherwise influence an opiate receptor. Endogenous opioid peptides function as physiological ligands for an opioid receptor. Endogenous opioid peptides are presently categorized into four different classes: enkephalins, endomorphins, dynorphins, and endorphins (Akil et al, (1984) Annu. Rev. Neurosci. 7: 223-255; De Luca et al., (1996) Pharmacol. Ther. 69: 103-115; Okada et al., (2002) Vitam. Horm. 65: 257-279; and Bodnar and Hadjimarkou, (2003) Peptides 24: 1241-1302). The structures of various endogenous opioid peptides are shown in Table 1.

TABLE 1
Endogenous oploid peptides and their precursors

| | | |
|---|---|---|
| Opioid peptide | Structure | Precursor |

| | | |
|---|---|---|
| Met-enkephalin | YGGFM | Proenkephalin |
| | (SEQ ID NO:3) | |
| | or | |
| Leu-enkephalin | YGGFL | Proenkephalin |
| | (SEQ ID NO:4) | |
| Octapeptide | YGGFMRGL | Proenkephalin |
| | (SEQ ID NO:5) | |
| Heptapeptide | YGGFMRF | Proenkephalin |
| | (SEQ ID NO:6) | |
| β-Endorphin | A peptide with 31 | Pro- |
| | amino acids | opiomelanocortin |
| Dynorphin 1-8 | YGGFLRRI | Prodynorphin |
| | (SEQ ID NO:7) | |
| Dynorphin 1-17 | YGGFLRRIRPKLKWDNQ | Prodynorphin |
| | (SEQ ID NO:8) | |
| A-Neoendorphin | YGGFLRKYPK | Prodynorphin |
| | (SEQ ID NO:9) | |
| B-Neoendorphin | YGGFLRKYP | Prodynorphin |
| | (SEQ ID NO:10) | |
| Endomorphin 1 | YPWF-NH2 | Not known |
| | (SEQ ID NO:11) | |
| Endomorphin 2 | YPFF-NH2 | Not known |
| | (SEQ ID NO:12) | |

The endogenous opioid peptide transport system of the present invention demonstrates one or more of the following functional activities.

The endogenous opioid peptide transport system of the present invention may demonstrate an upregulation in the transport of endogenous opioid peptide by the Tat protein encoded by the human immunodeficiency virus type I.

The endogenous opioid peptide transport system of the present invention may demonstrate transport of an endogenous opioid peptide that is coupled to a sodium gradient. The stoichiometry may be more than one Na⁺ ion for each endogenous opioid peptide transported.

The endogenous opioid peptide transport system of the present invention may demonstrate transport of an endogenous opioid peptide that is coupled to a chloride gradient. The stoichiometry may be one Cl⁻ ion for each endogenous opioid peptide transported.

The endogenous opioid peptide transport system of the present invention may demonstrate negative modulation or inhibition of the transport of endogenous opioid peptide by L-lysine, L-valine, D-alanine, D-tyrosine, L-arginine, analogs or structural derivatives thereof (i.e., L-lysine, L-valine, D-alanine, D-tyrosine, or L-arginine) and/or a combination thereof. As used herein, analogs or structurally derivatives thereof include amino acids modified, for example, by chemical and/or enzymatic derivatization, including side chain modifications, backbone modifications, and N— and C-terminal modifications including acetylation, hydroxylation, methylation, amidation, and the attachment of carbohydrate or lipid moieties, cofactors, and the like. Analogs or structural derivatives include, for example, the c-carboxy ester derivatives of L-lysine, L-valine, D-alanine, D-tyrosine, or L-arginine.

The endogenous opioid peptide transport system of the present invention may demonstrate positive modulation or stimulation of the transport of endogenous opioid peptide by the tripeptides Gly-Gly-Be, Gly-Gly-Phe, Gly-Gly-Gly, Try-Gly-Gly, Glu-Gly-Phe, and/or analog or structural derivatives thereof of each of these tripeptides. As used herein, analogs or structurally derivatives thereof include tripeptides modified, for example, by chemical and/or enzymatic derivatization, including side chain modifications, backbone modifications, and N— and C-terminal modifications including acetylation, hydroxylation, methylation, amidation, and the attachment of carbohydrate or lipid moieties, cofactors, and the like.

The present invention includes methods for identifying agents that serve as substrates, modifiers, stimulators or inhibitors for one or more functional activities of the endogenous opioid peptide transport system described herein.

As used herein a “modulator” or “modifier” of an endogenous opioid peptide transport system is an agent that alters the transmembrane transport of an endogenous opioid peptide via the endogenous opioid peptide transport system.

A modulator may be an activator or stimulator of an endogenous opioid peptide transport system. As used herein an “activator” or “stimulator” of an endogenous opioid peptide transport system is an agent that increases or enhances the transmembrane transport of an endogenous opioid peptide via the endogenous opioid peptide transport system. “Activators” are agents that increase, open, activate, facilitate, enhance activation, agonize, or up regulate an endogenous opioid peptide transport system. Examples of a stimulator of an endogenous opioid peptide transport system include, for example, the tripeptides Gly-Gly-Ile, Gly-Gly-Phe, Gly-Gly-Gly, Try-Gly-Gly, Glu-Gly-Phe, and/or analogs or structural derivatives of each of Gly-Gly-Ile, Gly-Gly-Phe, Gly-Gly-Gly, Try-Gly-Gly, or Glu-Gly-Phe.

A modulator may be an inhibitor of an endogenous opioid peptide transport system. As used herein an “inhibitor” of an endogenous opioid peptide transport system is an agent that decreases or reduces transmembrane transport of an endogenous opioid peptide via the endogenous opioid peptide transport system. Inhibitors are agents that, partially or totally block activity, decrease, prevent, delay activation, inactivate, or down regulate the activity or expression of an endogenous opioid peptide transport system. Examples of such an inhibitor include, for example, L-lysine, L-valine, D-alanine, D-tyrosine, L-arginine, and/or analogs or structural derivatives of each of L-lysine, L-valine, D-alanine, D-tyrosine, or L-arginine.

A modulator may blocker an endogenous opioid peptide transport system. As used herein a “blocker” of an endogenous opioid peptide transport system is an agent that binds to the an endogenous opioid peptide transport system and blocks the transmembrane transport of an endogenous opioid peptide via the endogenous opioid peptide transport system but is itself not transported via the endogenous opioid peptide transport system.

Suitable agents can include naturally occurring or synthetic ligands, antagonists, agonists, antibodies, antisense molecules, ribozymes, small chemical molecules and the like. Suitable agents can also include modified versions of an endogenous opioid peptide transport polypeptide or versions with altered activity.

Modifiers, stimulators, inhibitors, and blockers of an endogenous opioid peptide transport system may be identified using a variety of assays, including the various in vitro and in vivo assays described herein. Assays for such agents can include, for example, expressing an endogenous opioid peptide transport system in vitro, in cells, in cell membranes, or in vivo, applying putative modulator compounds, and then evaluating the functional effects on activity, as described above. A wide variety of methods may be used to study the transport of opioid peptides by the endogenous opioid peptide transport system of the present invention, including, but not limited to, the methods described herein and the methods described in Egelton et al., J Pharmaceutical Sci. 1998; 87(11):1433-1439. A wide variety of methods may be used for the identification of agents that modulate the transport of endogenous opioid peptides by the endogenous opioid peptide transport system of the present invention, including, but not limited to, the methods described herein and the methods described in Smirga and Torii, Proc. Natl. Acad. Sci. U.S.A. 2003; 100(26):15370-15375.

Samples or assays of an endogenous opioid peptide transport system that are treated with a potential activator, inhibitor, or modulator can be compared to control samples without the inhibitor, activator, or modulator to examine the extent of modification. Untreated control samples can be assigned a relative protein activity value of 100%. Inhibition of an endogenous opioid peptide transport system, for example, is achieved when the activity value relative to the control is 80%, preferably 50%, more preferably 25-0%. Activation of an endogenous opioid peptide transport system, for example, is achieved when the activity value relative to the control (untreated with activators) is 110%, more preferably 150%, more preferably 200-500% (i.e., two to five fold higher relative to the control), more preferably 1000-3000% higher.

Agents of the present invention that modulate one or more functions of an endogenous opioid peptide transport system have a variety of therapeutic applications and may be administered in methods of treating opiate-related disorders. For example, agents that modulate one or more functional activities of an endogenous opioid peptide transport system may be used in novel methods of pain relief and pain management. For example, an inhibitor of an endogenous opioid peptide transport system may increase the concentration of opioid peptides in the synapse of opioidergic neurons, thus decreasing pain perception. Such agents may be administered to a subject receiving a narcotic for pain relief, thus reducing the dosage of the narcotic needed for effective pain management and lessening the addictive impact of the pain-relieving narcotic.

Agents that modulate one or more functional activities of an endogenous opioid peptide transport system may be administered to treat the chronic pain associated with HIV infection, including, for example, HIV-associated neuropathy.

Agents that modulate one or more functional activities of an endogenous opioid peptide transport system may be administered in methods of treating irritable bowel syndrome (IBS). For example, agents that inhibit the transmembrane transport of an endogenous opioid peptide may be administered to treat irritable bowel syndrome (IBS) with diarrhea and agents that stimulate the transmembrane transport of an endogenous opioid peptide may be administered to treat irritable bowel syndrome (IBS) with constipation. Agents that modulate one or more functional activities of an endogenous opioid peptide transport system may be administered in methods of treating other gastrointestinal disorders, diarrhea, and constipation.

As used herein, “treating” a condition or a subject includes therapeutic, prophylactic, and diagnostic treatments. Treatment can be initiated before, during, or after the development of the condition to be treated.

The administration of narcotics, such as morphine, decreases bowel motility, resulting in constipation. Agents that stimulate the transmembrane transport of an endogenous opioid peptide may be administered to increase bowel motility, thus reducing or preventing the constipation associated with the administration of a narcotic.

One or more agents that modulate one or more functional activities of an endogenous opioid peptide transport system may be formulated as a composition. Such a composition may be formulated in any of a variety of forms adapted to the chosen route of administration. The formulations may be conveniently presented in unit dosage form and may be prepared by methods well known in the art of pharmacy. Formulations of the present invention may include, for instance, a pharmaceutically acceptable carrier. The formulations of this invention may include one or more accessory ingredients including diluents, buffers, binders, disintegrants, surface active agents, thickeners, lubricants, preservatives (including antioxidants) and the like. The formulations of this invention may further include additional therapeutic agents, for example, known analgesic agents and/or known anti-diarrheal agents.

The present invention includes isolated endogenous opioid peptide transporter polypeptides. “Polypeptide,” as used herein, refers to a polymer of amino acids and does not refer to a specific length of a polymer of amino acids. Thus, for example, the terms peptide, oligopeptide, protein, and enzyme are included within the definition of polypeptide, whether naturally occurring or synthetically derived, for instance, by recombinant techniques or chemically or enzymatically synthesized. This term also includes post-expression modifications of the polypeptide, for example, glycosylations, acetylations, phosphorylations, and the like. The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

An opioid peptide transporter polypeptide may demonstrate one or more of the functional activities of an endogenous opioid peptide transport system as described herein. Such functional activity of an endogenous opioid peptide transporter polypeptide can be easily assessed using the various assays described herein as well as other assays well known to one with ordinary skill in the art. A modulation in functional activity, including the stimulation or the inhibition of functional activity, can be readily ascertained by the various assays described herein, and by assays known to one of skill in the art.

A modulation in a functional activity can be quantitatively measured and described as a percentage of the functional activity of a comparable control. The functional activity of the present invention includes a modulation that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% of the activity of a suitable control.

For example, the stimulation of a functional activity can be quantitatively measured and described as a percentage of the functional activity of a comparable control. The functional activity of the present invention includes a stimulation that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% of the activity of a suitable control.

For example, inhibition of a functional activity can be quantitatively measured and described as a percentage of the functional activity of a comparable control. The functional activity of the present invention includes an inhibition that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% of the activity of a suitable control.

The present invention includes biologically active analogs of an endogenous opioid peptide transporter polypeptide. A “biologically active analog” of a polypeptide includes polypeptides having one or more amino acid substitutions. A biologically active analog of a polypeptide may retain one or more of the functional activities of the unsubstituted polypeptide. Substitutes for an amino acid in the polypeptides of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Examples of such preferred conservative substitutions include Lys for Arg and vice versa to maintain a positive charge; Glu for Asp and vice versa to maintain a negative charge; Ser for Thr so that a free —OH is maintained; and Gin for Asn to maintain a free NH2. Likewise, biologically active analogs of an opioid peptide transporter polypeptide containing deletions or additions of one or more contiguous or noncontiguous amino acids are also contemplated.

The biologically active analog of an endogenous opioid peptide transporter polypeptide of the present invention includes “fragments” and “modifications” of an opioid peptide transport system polypeptide. As used herein, a “fragment” of an opioid peptide transport system polypeptide means an opioid peptide transport system polypeptide that has been truncated at the N-terminus, the C-terminus, or both. A fragment may range from about 5 to about 250 amino acids in length. For example it may be 5, 10, 20, 25, 50, 75, 100, 125, 150, 175, 200, 225, or 250 amino acids in length. Fragments of an opioid peptide transport system polypeptide with potential biological activity can be identified by many means. One means of identifying such fragments of an opioid peptide transport system polypeptide with biological activity is to compare the amino acid sequences of an opioid peptide transport system polypeptide from rat, mouse, human and/or other species to one another. Regions of homology can then be prepared as fragments. Fragments of a polypeptide also include a portion of the polypeptide containing deletions or additions of one or more contiguous or noncontiguous amino acids. The resulting polypeptides may retain one or more of the biological activities of the full-length polypeptide or may exhibit a reduction or increase in one or more of these activities.

A “modification” of an endogenous opioid peptide transporter polypeptide includes endogenous opioid peptide transporter polypeptides or fragments thereof that have been chemically or enzymatically derivatized at one or more constituent amino acid, including side chain modifications, backbone modifications, and N— and C-terminal modifications including acetylation, hydroxylation, methylation, amidation, and the attachment of carbohydrate or lipid moieties, cofactors, and the like. Modified polypeptides of the invention may retain one or more of the biological activities of the unmodified polypeptide or may exhibit a reduction or increase in one or more of these activities.

The polypeptides and biologically active analogs thereof of the present invention include native (naturally occurring), recombinant, and chemically or enzymatically synthesized polypeptides. For example, the opioid peptide transport system polypeptides of the present invention may be prepared by isolation form naturally occurring tissues or prepared recombinantly, by well known methods, including, for example, preparation as fusion proteins in bacteria and insect cells.

The polypeptides of the present invention include polypeptides with “structural similarity” to naturally occurring polypeptides. As used herein, “structural similarity” refers to the identity between two polypeptides. For polypeptides, structural similarity is generally determined by aligning the residues of the two polypeptides to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of opioid peptide transport system protein sequences can carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the world wide web at ncbi.nlm.nih.gov/BLAST/. The default values for all BLAST 2 search parameters may be used, including matrix =BLOSUM62; open gap penalty=11, extension gap penalty=1, gap x-dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids and “similarity” refers to the presence of not only identical amino acids but also the presence of conservative substitutions.

The opioid peptide transport system polypeptides of the present invention include polypeptides with at least 35%, at least 40%, at least 45%, at least 50%, at least 55%, at least 60%, at least 65%, at least 70%, at least 75%, at least 80%, at least 85%, at least 90%, at least 95%, or at least 99% sequence similarity to a known endogenous opioid peptide transporter polypeptide.

Amino acids essential for the function of opioid peptide transport system polypeptides can be identified according to procedures known in the art, such as site-directed mutagenesis or alanine-scanning mutagenesis (Cunningham and Wells, Science 244: 1081-1085, 1989; Bass et al., Proc. Natl. Acad. Sci. USA 88: 4498-4502, 1991).

The present invention includes isolated polynucleotides encoding endogenous opioid peptide transporter polypeptides. Such endogenous opioid peptide transporter polypeptides may demonstrate one or more of the functional activities of endogenous opioid peptide transport system described herein.

“Polynucleotide” and “nucleic acid sequences” are used interchangeably to refer to a linear polymeric form of nucleotides of any length, either ribonucleotides or deoxynucleotides, and includes both double- and single-stranded DNA and RNA. A polynucleotide can be linear or circular in topology. A polynucleotide can be obtained using any method, including, without limitations, common molecular cloning and chemical nucleic acid synthesis. A polynucleotide may include nucleotide sequences having different functions, including for instance coding sequences, and non-coding sequences.

Also included in the present invention are polynucleotides hybridizing to a polynucleotide encoding an endogenous opioid peptide transporter polypeptide, or a complement thereof, under standard hybridization conditions, that encode a polypeptide that exhibits one or more of the functional activities of an endogenous opioid peptide transporter.

As used herein, “stringent hybridization conditions” refer to hybridization conditions such as 6×SSC, 5× Denhardt, 0.5% sodium dodecyl sulfate (SDS), and 100 μg/ml fragmented and denatured salmon sperm DNA hybridized overnight at 65° C. and washed in 2×SSC, 0.1% SDS at least one time at room temperature for about 10 minutes followed by at least one wash at 65° C. for about 15 minutes followed by at least one wash in 0.2×SSC, 0.1% SDS at room temperature for at least 3-5 minutes. Typically, a 20×SSC stock solution contains about 3M sodium chloride and about 0.3M sodium citrate.

As used herein, “complement” and “complementary” refer to the ability of two single stranded polynucleotides to base pair with each other, where an adenine on one polynucleotide will base pair to a thymine on a second polynucleotide and a cytosine on one polynucleotide will base pair to a guanine on a second polynucleotide. Two polynucleotides are complementary to each other when nucleotide sequences in a polynucleotide can base pair with a nucleotide sequence in a second polynucleotide. For instance, 5′-ATGC and 5′-GCAT are complementary. Typically two polynucleotides are complementary if they hybridize under the standard conditions referred to herein.

As used herein, the term “isolated” means that a polynucleotide or polypeptide is either removed from its natural environment or synthetically derived, for instance by recombinant techniques, or chemically or enzymatically synthesized. An isolated polynucleotide denotes a polynucleotide that has been removed from its natural genetic milieu and is thus free of other extraneous or unwanted coding sequences, and is in a form suitable for use within genetically engineered protein production systems. Isolated polynucleotides of the present invention are free of other coding sequences with which they are ordinarily associated, but may include naturally occurring 5′ and 3′ untranslated regions such as promoters and terminators. Preferably, the polynucleotide or polypeptide is purified, i.e., essentially free from any other polynucleotides or polypeptides and associated cellular products or other impurities.

As used herein “coding sequence,” “coding region,” and “open reading frame” are used interchangeably and refer to a polynucleotide that encodes a polypeptide, usually via mRNA, when placed under the control of appropriate regulatory sequences. The boundaries of the coding region are generally determined by a translation start codon at its 5′ end and a translation stop codon at its 3′ end.

Also included in the present invention are polynucleotides having a sequence identity of at least 50%, at least 55%, at least 60%, at least 65%, at least 70%, at least 75%, at least 80%, at least 85%, at least 90%, at least 95%, at least 96%, at least 97%, at least 98%, or at least 99% with the nucleotide sequence of polynucleotide encoding an endogenous opioid peptide transporter polypeptide, where the polynucleotide encodes a polypeptide that exhibits one or more of the functional activities of an endogenous opioid peptide transport system described herein.

As used herein, “sequence identity” refers to the identity between two polynucleotide sequences. Sequence identity is generally determined by aligning the residues of the two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate sequence is the sequence being compared to a known sequence. For example, two polynucleotide sequences can be compared using the Blastn program of the BLAST 2 search algorithm, as described by Tatiana et al., FEMS Microbiol Lett., 1999;174: 247-250, and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x-dropoff=50, expect=10, wordsize=11, and filter on.

Also included in the present invention are polynucleotide fragments. A polynucleotide fragment is a portion of an isolated polynucleotide as described herein. Such a portion may be several hundred nucleotides in length, for example 100, 200, 300, 400, 500, 600, 700, 800, 900 or 1000 nucleotides in length. Such a portion may be 10 nucleotides to 100 nucleotides in length, including but not limited to, 14 to 40 nucleotides in length.

The polynucleotides of the present invention may be formulated in a composition along with a “carrier.” As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

By “pharmaceutically acceptable” is meant a material that is not biologically or otherwise undesirable, i.e., the material may be administered to an individual along with a polynucleotide encoding an opioid peptide transport system polypeptide without causing any undesirable biological effects or interacting in a deleterious manner with any of the other components of the pharmaceutical composition in which it is contained.

Polynucleotides of the present invention can be inserted into a vector. Construction of vectors containing a polynucleotide of the invention employs standard ligation techniques known in the art. See, for instance, Sambrook et al, “Molecular Cloning: A Laboratory Manual,” Cold Spring Harbor Laboratory Press, 1989. The term vector includes, but is not limited to, plasmid vectors, viral vectors, cosmid vectors, or artificial chromosome vectors. Typically, a vector is capable of replication in a bacterial host, for instance, E. coli. Selection of a vector depends upon a variety of desired characteristics in the resulting construct, such as a selection marker, vector replication rate, and the like. A vector can provide for further cloning (amplification of the polynucleotide), e.g., a cloning vector, or for expression of the polypeptide encoded by the coding sequence, e.g., an expression vector. Suitable host cells for cloning or expressing the vectors herein are prokaryote or eukaryotic cells.

As used herein, an “expression vector” is a DNA molecule, linear or circular, that includes a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

By “host cell” is meant a cell that supports the replication or expression of an expression vector. Host cells may be bacterial cells, including, for example, E. coli and B. subtilis, or eukaryotic cells, such as yeast, including, for example, Saccharomyces and Pichia, insect cells, including, for example, Drosophila cells and the Sf9 host cells for the baculovirus expression vector, amphibian cells, including, for example, Xenopus oocytes and mammalian cells, such as CHO cells, HeLa cells, human retinal pigment epithelial (RPE) cells, human hepatoma HepG2 cells, and plant cells.

An expression vector optionally includes regulatory sequences operably linked to the coding sequence. The invention is not limited by the use of any particular promoter, and a wide variety of promoters are known. Promoters act as regulatory signals that bind RNA polymerase in a cell to initiate transcription of a downstream (3′ direction) coding sequence. The promoter used can be a constitutive or an inducible promoter. It can be, but need not be, heterologous with respect to the host cell.

The transformation of a host cell with an expression vector may be accomplished by a variety of means known to the art, including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

Transformation of a host cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) that detects the presence of a polypeptide encoded by one or more of the transgenes. Alternatively, transient transformation may be detected by detecting the activity of the protein encoded by the transgene. The term “transient transformant” refers to a cell that has transiently incorporated one or more transgenes. In contrast, the term “stable transformation” or “stably transformed” refers to the introduction and integration of one or more transgenes into the genome of a cell. The term “stable transformant” refers to a cell that has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Methods for both transient and stable expression of coding regions are well known in the art.

Among the known methods for expressing transporter genes is expression in a Xenopus oocyte system. A cDNA encoding the open reading frame of a citrate transporter polypeptide or portions thereof can be incorporated into commercially available bacterial expression plasmids such as the pGEM (Promega) or pBluescript (Stratagene) vectors or one of their derivatives. After amplifying the expression plasmid in bacterial (E. coli) cells the DNA is purified by standard methods. The incorporated transporter sequences in the plasmid DNA are then transcribed in vitro according to standard protocols, such as transcription with SP6 or T7 RNA polymerase. The RNA thus prepared is injected into Xenopus oocytes where it is translated and the resulting transporter polypeptides are incorporated into the plasma membrane. The functional properties of these transporters can then be investigated by electrophysiological, biochemical, pharmacological, and related methods.

The polynucleotides of the present invention may be inserted into a recombinant DNA vector for the production of products including, but not limited to, mRNA, antisense oligonucleotides, and polynucleotides for use in RNA interference (RNAi) (see, for example, Cheng et al., Mol Genet Metab. (2003);80: 121-28). For example, for the production of mRNA a DNA sequence, or fragments thereof, may be inserted into a plasmid containing a promoter for either SP6 or T7 RNA polymerase. The plasmid is cut with a restriction endonuclease to allow run-off transcription of the mRNA, and the RNA is produced by addition of the appropriate buffer, ribonucleotides, and polymerase. The RNA is isolated by conventional means such as ethanol precipitation. The mRNA can be capped or polyadenylated, for example, prior to injection into a cell such as a Xenopus oocyte, for expression.

The present invention also includes transgenic and knockout animal models, useful in studies to further understand the physiological functions of this transporter. A transgenic or knockout animal is preferably a mammal, for example a rodent, such as a rat or mouse. Other examples include non-human primates, sheep, dogs, cows, goats, chickens, amphibians, and fish. A knockout animal may be an animal with a knockout mutation in one or more alleles encoding an endogenous opioid peptide transporter of the present invention. Transgenic and knockout animals may be constructed using standard methods known in the art and as set forth, for example, in U.S. Pat. Nos. 5,614,396 5,487,992, 5,464,764, 5,387,742, 5,347,075, 5,298,422, 5,288,846, 5,221,778, 5,175,384, 5,175,383, 4,873,191, 4,736,866, and Moles et al., Science (2004) 304:1983-1986.

Included in the present invention are antibodies that specifically bind to one or more of the polypeptides described herein. Such antibodies include, but are not limited to, polyclonal antibodies, affinity-purified polyclonal antibodies, monoclonal antibodies, humanized antibodies, chimeric antibodies, anti-idiotypic antibodies, single chain antibodies, and antigen-binding fragments thereof, such as F(ab′)₂ and Fab proteolytic fragments and fragments produced from an Fab expression library. The term “polyclonal antibody” refers to an antibody produced from more than a single clone of plasma cells; in contrast “monoclonal antibody” refers to an antibody produced from a single clone of plasma cells.

As used herein, “antibodies” or “antibody” refers to an immunoglobulin molecule or immunologically active antigen-binding portion thereof. In preferred embodiments, an antibody has at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al., J. Mol. Biol. 1987;196: 901-917). Each VH and VL is composed of three CDR's and four FRs, arranged from amino-terminus to carboxy-terminus in the following order: FR1, CDR1, FR2, CDR2, FR3, CDR3, FR4.

The phrase “specifically binds” or “specifically immunoreactive with,” when referring to an antibody, refers to a binding reaction that is determinative of the presence of a protein in a heterogeneous population of proteins and other biologics. Thus, under designated immunoassay conditions, the specified antibodies bind to a particular protein at least two times the background and do not substantially bind in a significant amount to other proteins present in the sample. Typically a specific or selective reaction will be at least twice background signal or noise and more typically more than 10 to 100 times background. Specific binding to an antibody under such conditions may require an antibody that is selected for its specificity for a particular protein Antibodies of the present invention can be prepared using the intact polypeptide or fragments thereof as the immunizing agent. If a polypeptide fragment is used as an immunizing agent, a preferred fragment is about 15 to about 30 contiguous amino acids. For example, contiguous amino acid fragments of 14, 15, 16, 17, 18, 19, 20, 21, 22, 23, 24, 25, 26, 27, 28, 29, 30, 31, or 32 amino acids may be used.

In addition to specifically binding to an opioid peptide transport system polypeptide, the antibodies may have additional binding specificities. For example, an antibody may bind to the C terminus or the N terminus of an opioid peptide transport system polypeptide. Or, an antibody may be selected that demonstrates limited cross reactivity. For example, an antibody may bind to a human opioid peptide transport system polypeptide, but not to a rat opioid peptide transport system polypeptide or mouse opioid peptide transport system polypeptide.

The preparation of polyclonal antibodies is well known. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. The resulting antibodies may be isolated from other proteins by using an affinity column having an Fc binding moiety, such as protein A, or the like.

Monoclonal antibodies can be obtained by various techniques familiar to those skilled in the art. Briefly, spleen cells from an animal immunized with a desired antigen are immortalized, commonly by fusion with a myeloma cell (see, for example, Kohler and Milstein, Eur. J. Immunol. (1 976);6: 511-519; J. Goding (1986) In “Monoclonal Antibodies: Principles and Practice,” Academic Press, pp 59-103; and Harlow et al., Antibodies: A Laboratory Manual, page 726 (Cold Spring Harbor Pub. 1988). Monoclonal antibodies can be isolated and purified from hybridoma cultures by techniques well known in the art.

In some embodiments, the antibody can be recombinantly produced, for example, produced by phage display or by combinatorial methods. Phage display and combinatorial methods can be used to isolate recombinant antibodies that bind to an opioid peptide transporter polypeptide or fragments thereof (see, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology (1991);9: 1370-1372; Huse et al., Science (1989);246: 1275-1281; Griffths et al., EMBO J. (1993);12: 725-734; Hawkins et al., J Mol Biol (1992);226: 889-896; Clackson et al., Nature (1991);352: 624-628; Gram et al., PNAS (1992);89:3576-3580; Garrad et al., Bio/Technology (1991);9: 1373-1377; Hoogenboom et al., Nuc Acid Res (1991);19: 4133-4137; and Barbas et al., PNAS (1991);88: 7978-7982). Such methods can be used to generate human monoclonal antibodies.

Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, for example, WO 91/00906; WO 91/10741; WO 92/03918, Lonberg et al., Nature (1994);368: 856-859; Green et al., Nature Genet. (1994);7: 13-21; Morrison et al., PNAS (1994);81: 6851-6855; Tuaillon et al., PNAS (1993);90:3720-3724; Bruggeman et al., Eur J Immunol (1991);21:1323-1326).

A therapeutically useful antibody may be derived from a “humanized” monoclonal antibody. Humanized monoclonal antibodies are produced by transferring one or more CDRs from the heavy and light variable chains of a mouse (or other species) immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found, for example, in Jones et al., Nature (1986);321: 522 and Singer et al., J. Immunol., (1993);150: 2844.

In addition, chimeric antibodies can be obtained by splicing the genes from a mouse antibody molecule with appropriate antigen specificity together with genes from a human antibody molecule of appropriate biological specificity; see, for example, Takeda et al., Nature (1985);314: 544-546. A chimeric antibody is one in which different portions are derived from different animal species.

Antibody fragments can be generated by techniques well known in the art. Such fragments include Fab fragments produced by proteolytic digestion, and Fab fragments generated by reducing disulfide bridges.

Antibodies, or fragments thereof, may be coupled directly or indirectly to a detectable marker by techniques well known in the art. A detectable marker is an agent detectable by spectroscopic, photochemical, biochemical, immunochemical, or chemical means. For example, useful detectable markers include fluorescent dyes, chemiluminescent compounds, radioisotopes, electron-dense reagents, enzymes, colored particles, biotin, or dioxigenin. A detectable marker often generates a measurable signal, such as radioactivity, fluorescent light, color, or enzyme activity.

When used for immunotherapy, antibodies, or fragments thereof, may be unlabelled or labeled with a therapeutic agent. These agents can be coupled directly or indirectly to the monoclonal antibody by techniques well known in the art, and include such agents as drugs, radioisotopes, lectins and toxins. Antibodies can be used alone or in combination with additional therapeutic agents, such as those described above. Preferred combinations include monoclonal antibodies with modifiers of citrate transporters or other biological response modifiers. The dosage administered may vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, subcutaneously, intracavity, or transdermally, alone or with effector cells.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Identification of a Novel Na⁺- and Cl⁻-Coupled Transport System for Endogenous Opioid Peptides in Retinal Pigment Epithelium and Induction of the Transport System by HIV-1 Tat

In this example a new peptide transport system in the human retinal pigment epithelial (RPE) cells is identified that transports a variety of endogenous opioid peptides with high affinity. This hitherto unrecognized transport system was identified when analysing the differential effects of Tat, the transacting factor encoded by HIV-1, on various transport processes in RPE cells. This transport system is markedly induced by Tat. This opioid transport system is energized by transmembrane Na⁺ and Cl⁻ gradients and is distinct from any of the previously identified transport systems for opioid peptides in mammalian cells. Free amino acids, dipeptides, tripeptides and non-peptide opiate receptor antagonists are excluded by this newly identified transport system. The affinities of endogenous opioid peptides for this system are in the range of 0.4-40 micromolar (EM). The identification of the high-affinity Na⁺- and Cl⁻-coupled transport system in mammalian cells that is specific for endogenous opioid peptides and is induced by HIV-1 Tat is of significance not only to the biology of opioid peptides but also to the pathology of HIV-1 infection in humans.

HIV-1 genome-encoded Tat is the major transactivator of the virus expression and is released into the circulation of persons with HIV-1 infection via secretion by, or lysis of, infected cells. HIV-1 Tat exerts a variety of biological effects on mammalian cells (Gaynor, Curr. Top. Microbiol. Immunol., 1995;193:51-77; Gallo, Proc. Natl. Acad. Sci. U.S.A., 1999;96:8324-8326). Recently, studies to investigate the influence of HIV-1 Tat on gene expression in RPE were initiated, with special emphasis on the expression of the genes coding for membrane transporters. One of the important functions of RPE is to mediate the transcellular transfer of nutrients from choroidal blood into the subretinal space to nourish the photoreceptor cells (Hughes et al., (1998) “Transport mechanisms in retinal pigment epithelium.” In The Retinal Pigment Epithelium: Current Aspects of Function and Disease (Marmor, M. F. and Wolfensberger, T. J., eds.), pp. 103-134, Oxford University Press, Oxford).

In this example, HIV-1 Tat protein was stably expressed in the human RPE cell line ARPE-19 and monitored the differential expression of genes in control ARPE-19 cells and in Tat-expressing ARPE cells by microarray analysis. It was found that expression of several transporter genes was affected markedly by HIV-1 Tat. This included the up-regulation of the genes coding for the amino acid transporter B⁰ (ATB⁰) and the light chain of the amino acid transport system x⁻_{c} (xCT) and for the creatine transporter and the down-regulation of the gene coding for the organic anion transporting polypeptide (OATP-A). In the case of the up-regulated genes, microarray data was corroborated with functional analysis by demonstrating an increase in the transport activity for ATB⁰, x⁻_{c} (the transport system consisting of xCT as the light chain), and creatine transporter in Tat-expressing ARPE cells compared with control cells stably transfected with vector alone (Ganapathy et al., Invest. Ophthalmol. Vis. Sci., 2002;43:E-abstract 4565; Hu et al., Invest. Ophthalmol. Vis. Sci., 2003;44:E-abstract 4616; and Ganapathy et al., Invest. Ophthalmol. Vis. Sci. 2003;44:E-abstract 2275).

Studies to analyze the transport function for the down-regulated OATP-A were intiated. Since OATP-A transports opioid peptides such as [D-penicillamine^{2,5}]enkephalin (DPDPE), deltorphin II, and Leu-enkephalin (Gao et al., J. Pharmacol. Exp. Ther., 2000;294:73-79), deltorphin II was used as the substrate in transport assays in an attempt to confirm the microarray data, expecting a decrease in the transport activity in Tat-expressing cells compared with control cells. Surprisingly, the transport of this opioid peptide was many-fold higher in Tat-expressing ARPE-19 cells than in control cells. Furthermore, while the transport of deltorphin II via OATP-A occurs via a Na⁺-independent process (Gao et al., J. Pharmacol. Exp. Ther., 2000;294:73-79), it was found that the transport of this peptide in control and Tat-expressing ARPE-19 cells to be obligatorily dependent on the presence of Na⁺ and Cl⁻. Substrate specificity studies have shown that the transport of deltorphin II in these cells is effectively inhibited by a variety of endogenous opioid peptides consisting of 4-13 amino acids. This is the first evidence for the existence of a Na⁺- and Cl⁻-coupled active transport system for endogenous opioid peptides in mammalian cells. The identification of this novel transport system and the findings that this transport system is up regulated by HIV-1 Tat have important physiological and clinical implications.

Vectors, antibodies, opioid peptides and the opioid receptor antagonists. The HIV-1 Tat cDNA construct in pGEM2 vector (catalogue no. 909) and the monoclonal antibody to HIV-1 Tat (catalogue no. 4138) were obtained from the NIH AIDS Research and Reference Reagent Program (Rockville, Md., U.S.A.). The human RPE cell line ARPE-19 was obtained from the American Type Culture Collection (Manassas, Va., U.S.A.). FITC-conjugated goat anti-mouse IgG was from Jackson ImmunoResearch Laboratories (West Grove, Pa., U.S.A.). Unlabelled opioid peptides and the opioid receptor antagonists (naloxone and naltrexone) were purchased from Sigma Chemicals (St. Louis, Mo., U.S.A.). Tyr-D-Ala²-[3,5-³H]deltorphin II (specific radioactivity 38.5 Ci/mmol; referred to subsequently as [³ H]deltorphin II) was obtained from PerkinElmer Life Sciences, Inc. (Boston, Mass., U.S.A.).

Stable transfection of ARPE-19 cells with pcDNA-Tat cDNA. The pGEM2-Tat cDNA construct contained as the insert a 295 basepair (bp) fragment encoding the first exon of HIV-1 Tat gene joined to a 145 bp pET3A transcription terminator. The entire insert (440 bp) was removed from the construct by digestion with PstI and EcoRI and ligated into the multiple cloning region of pcDNA3.1 (−) vector at PstI/EcoRI site so that the insert is downstream of the cytomegalovirus (CMV) promoter. The resultant construct was sequenced to confirm the orientation of the insert. This construct was electroporated into ARPE-19 cells and the stably transfected cell clones were isolated in the presence of Geneticin (G418). To serve as a control, empty pcDNA3.1 vector was electroporated into ARPE-19 cells in an identical manner and the clones harboring the vector were selected by G418 resistance.

The expression of HIV-1 Tat in the stably transfected cell line (Tat-ARPE-19) was confirmed by the analysis of Tat mRNA and Tat protein. Total RNA was isolated from control ARPE-19 cells and Tat-ARPE-19 cells and used as the template for a reverse transcriptase polymerase chain reaction (RT-PCR) to monitor the expression of Tat mRNA. The primers used for RT-PCR were: 5′-GTCAACATAGCAGAATAGGCAT-3′ (SEQ ID NO:1) (sense) and 5′GTACCCATCCGGATATAGTTC-3′ (SEQ ID NO:2) (antisense). These primers encompassed the entire insert in the pcDNA-Tat construct and the expected size of the RT-PCR product was 441 bp. The expression of the Tat protein in the stably transfected cell line was monitored by immunofluorescence using a monoclonal antibody specific for HIV-1 Tat and a FITC-conjugated secondary antibody.

Uptake measurements in control ARPE-19 cells and Tat-ARPE-19 cells. Control ARPE-19 cells and Tat-ARPE-19 cells were maintained in 75-cm² culture flasks in Dulbecco's modified Eagle's medium/F₁₂ medium (1:1, volume/volume (v/v)) in the presence of fetal bovine serum (10%) and G418 (100 μg/ml). For uptake measurements, cells were released by trypsin treatment and seeded in 24-well culture plates at an initial density of 0.1×10⁶ cells/well. The culture medium was replaced with fresh medium on the second day following the initial seeding and uptake measurements were made on the third day. The medium was removed by aspiration and uptake buffer containing [³H]deltorphin II was added to the cells to initiate uptake. After incubation at 37° C. for a desired time, uptake was terminated by the removal of the medium and washing of the cells with ice-cold uptake buffer. The cells were then dissolved in 1% SDS in 0.2 M NaOH and used for measurement of radioactivity. The uptake buffer in most experiments was 25 mM Hepes/Tris (pH 7.5), containing 140 mM NaCl, 5.4 mM KCl, 1.8 mM CaCl₂, 0.8 mM MgSO₄ and 5 mM glucose.

When the influence of Na⁺ on the uptake process was investigated, the concentration of NaCl in the uptake buffer was adjusted, as desired, by isoosmotically replacing NaCl with N-methyl-D-glucamine chloride. To assess the influence of Cl⁻ on the uptake process, the composition of the uptake medium was modified by replacing KCl and CaCl₂ with equimolar concentrations of potassium gluconate and calcium gluconate and the concentration of NaCl was adjusted, as desired, by iso-osmotically replacing NaCl with sodium gluconate. Saturation kinetics were analysed by fitting the data to the Michaelis-Menten equation. The Michaelis-Menten constant, Kₜ, was calculated by non-linear regression analysis and then confirmed by linear regression. Na⁺- and Cl⁻-activation kinetics were analyzed by fitting the data to the Hill equation and the Hill coefficients for Na⁺ and Cl⁻ (h; the number of Na⁺ and Cl⁻ ions involved in the activation process) and the K_{0.5} values (the concentration of Na⁺ and Cl⁻-needed for half-maximal activation) were determined from the analysis. Again, these constants were first calculated by non-linear regression methods and subsequently confirmed by linear regression methods. Experiments were repeated three times in duplicate and the results are given as means±S.E.M.

Evidence for the expression of HIV-1 Tat in ARPE-19 cells stably transfected with pcDNA-Tat construct. Control ARPE-19 cells (i.e., cells stably transfected with pcDNA vector alone) and Tat-ARPE- 19 cells were compared for expression of HIV-1 Tat by RT-PCR and immunofluorescence. RT-PCR was performed using total RNA isolated from control and Tat-ARPE-19 cells as the template and primers specific for the cDNA insert in pcDNA-Tat construct. An RT-PCR product of expected size (441 bp) was obtained only with RNA prepared from Tat-ARPE-19 cells. RNA from control cells did not yield any product. The expression of the Tat protein was analysed by immunofluorescence using a monoclonal antibody specific for Tat. The protein was detectable in Tat-ARPE-19 cells but not in control cells. These data demonstrate that ARPE-19 cells stably transfected with pcDNA-Tat construct express the HIV-1 Tat protein.

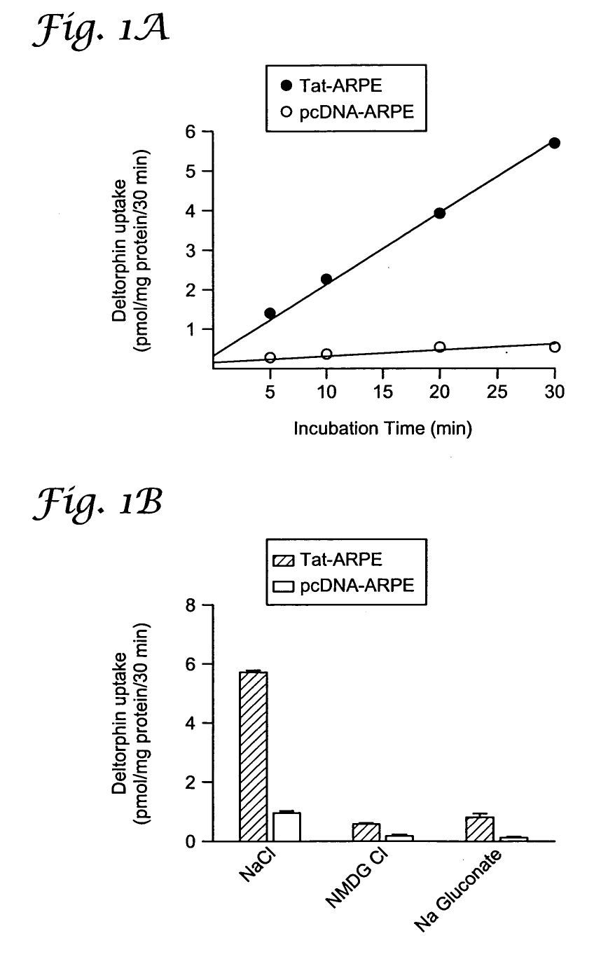

Deltorphin II uptake in control ARPE-19 cells and in Tat-ARPE-19 cells. FIG. 1A describes the time course of deltorphin II uptake in control ARPE-19 cells and in Tat-ARPE-19 cells. When measured in the presence of NaCl, deltorphin uptake was linear up to 30 minutes in both cell lines. But, the uptake in Tat-expressing cells was approximately 6-10-fold higher than in control cells. The uptake in both cell lines was obligatorily dependent on Na⁺ as well as Cl⁻ (FIG. 1B). Removal of Na⁺ or Cl⁻ from the uptake medium resulted in an 85-95% decrease in deltorphin uptake in control cells and in Tat-expressing cells. These data show that the deltorphin uptake detected in ARPE-19 cells occurs via a Na⁺- and Cl⁻-dependent process irrespective of whether or not the cells express HIV-1 Tat. It can be concluded that ARPE-19 cells express a Na⁺- and Cl⁻-dependent transport system for deltorphin II and that the expression of HIV-1 Tat leads to the up-regulation of this transport system.

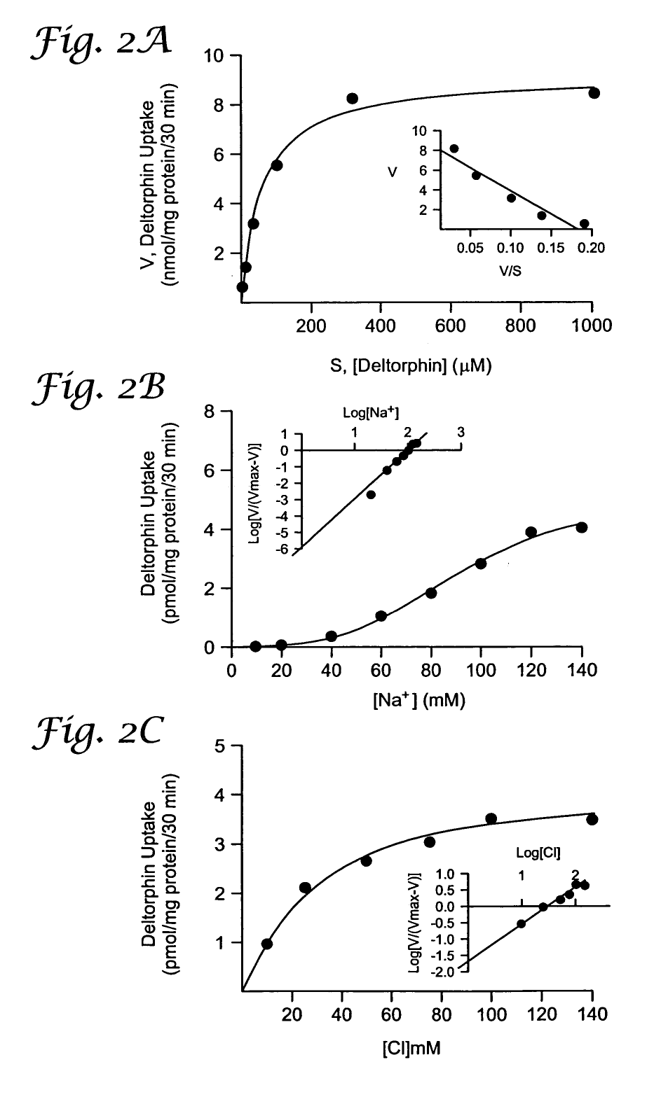

Kinetic characteristics of the deltorphin transport system. Since the Tat-expressing cells exhibit much higher activity of the deltorphin transport system than the control cells, the former were used to characterize the saturation kinetics and Na⁺- and Cl⁻-activation kinetics of the transport system. In the presence of NaCl, deltorphin II uptake was saturable with a Michaelis-Menten constant (Kₜ) of 46±5 μM (FIG. 2A). With the concentration of Cl⁻ kept constant at 140 mM, increasing concentrations of Na⁺ increased the uptake in a sigmoidal manner (FIG. 2B). The Hill coefficient (h) for the Na⁺-activation process was 3.0±0.7. With the concentration of Na⁺ kept constant at 140 mM, increasing concentrations of Cl⁻ also increased the uptake, but the relationship between the uptake rate and Cl⁻ concentration was hyperbolic (FIG. 2C). The value for the Hill coefficient (h) for the Cl⁻-activation process was 1.2±0.5. These data show that the Na⁺:Cl⁻:deltorphin stoichiometry for the transport process is 2 or 3:1:1.

Substrate specificity of the deltorphin transport system. The substrate selectivity of the deltorphin II uptake process was first investigated in Tat-expressing ARPE-19 cells by competition studies (Table 2). The uptake of [³ H]deltorphin II (50 nM) was measured in the absence and presence of a wide variety of opioid peptides and related compounds (1 mM). Free amino acids (L-tyrosine, L-proline and glycine), dipeptides (carnosine and Tyr-Pro), and tripeptides (Gly-Gly-Gly) did not compete with deltorphin for the uptake process to any significant extent. In fact, the tripeptide Gly-Gly-Gly caused a significant stimulation of deltorphin uptake (P<0.05). Interestingly, D-tyrosine inhibited the uptake by about 50%. The most interesting aspect of substrate selectivity of the transport system is that almost all of the endogenous opioid peptides tested showed marked competition with deltorphin for the uptake process. These peptides consisted of 4-13 amino acids. The only exceptions were β-lipotropin (Tyr-Gly-Gly-Phe) and tyrosine melanocyte-stimulating hormone inhibitory factor 1 (Tyr-MIF-1; Tyr-Pro-Leu-Gly-NH₂), which showed no or little inhibition of deltorphin uptake. As expected, unlabelled deltorphin II competed with radiolabeled deltorphin II for the uptake process. The other opioid peptides that showed marked inhibition of deltorphin uptake included Leu-enkephalin, Met-enkephalin, Met-enkephalinamide, [des-Tyr¹]Met-enkephalin, Met-enkephalin extended at the C-terminus by two or three amino acids, a-neo-endorphin, and various forms of dynorphin. Of note are the findings that naloxone and naltrexone, two of the well known non-peptide opiate antagonists, did not compete with deltorphin for the uptake process. These data show that the deltorphin transport system identified in Tat-ARPE-19 cells preferentially recognize endogenous opioid peptides as substrates. This unique substrate selectivity of the transport system is also seen in control ARPE-19 cells (Table 3).

Relative affinities of endogenous opioid peptides for the transport system. To determine the relative affinities of various endogenous opioid peptides for the transport system, the dose-response relationship for the inhibition of the uptake of [³H]deltorphin II by these peptides in Tat-ARPE-19 cells was investigated. The IC₅₀ values, calculated from these dose-response curves, are given in Table 4. Dynorphin A containing 13 amino acids showed highest affinity for the transport system with an IC₅₀ value of 0.38±0.03 μM. This was followed by [Arg⁶,Gly⁷,Leu⁸]Met-enkephalin, dynorphin B(1-9), dynorphin A(1-6), dynorphin A(1-7), [Arg⁶,Phe⁷]Met-enkephalin, Leu-enkephalin, Met-enkephalinamide, [des-Tyr¹]Met-enkephalin, Met-enkephalin, and deltorphin II. The IC₅₀ values for these peptides were in the range of 2.5×40 μM.

TABLE 2

Substrate selectivity of the deltorphin II trans-
port system in Tat-ARPE-19 cells.

| | | |
|---|---|---|
| | [³H] Deltorphin | |
| | II uptake | |
| | (pmol/mg | |
| Inhibitor (Structure) | protein/30 min) | (%) |

| | | |
|---|---|---|
| Control | 7.76 ± 0.27 | 100 |
| Gly-Gly-Gly | 9.68 ± 0.39* | 125 |
| L-Tyrosine | 7.52 ± 0.15 | 97 |
| L-Proline | 7.31 ± 0.20 | 94 |
| Glycine | 7.23 ± 0.14 | 93 |
| Carnosine (β-Ala-His) | 6.21 ± 0.08* | 80 |
| Tyr-Pro | 5.72 ± 0.09* | 74 |
| D-Tyrosine | 4.05 ± 0.09* | 52 |
| Tyr-MIF-1 (Tyr-Pro-Leu-Gly-NH₂) | 9.20 ± 0.15* | 119 |
| β-Lipotropin (Tyr-Gly-Gly-Phe) | 6.26 ± 0.31* | 81 |
| β-Casomorphin (Tyr-Pro-Phe-Pro- | 2.03 ± 0.05* | 26 |
| Gly-Pro-Ile) | | |
| Deltorphin II (Tyr-D-Ala-Phe- | 0.34 ± 0.00* | 4 |
| Glu-Val-Val-Gly-NH₂) | | |
| [des-Tyr¹] Met-enkephalin (Gly- | 0.74 ± 0.03* | 10 |
| Gly-Phe-Met) | | |
| Met-enkephalin (Tyr-Gly-Gly- | 0.50 ± 0.01* | 6 |
| Phe-Met) | | |
| Met-enkephalinamide (Tyr-Gly- | 0.69 ± 0.01* | 9 |
| Gly-Phe-Met-NH₂) | | |
| Leu-enkephalin (Tyr-Gly-Gly- | 0.15 ± 0.01* | 2 |
| Phe-Leu) | | |
| α-Neo-endorphin (1-6) | 0.19 ± 0.01* | 3 |
| (Tyr-Gly-Gly-Phe-Met-Lys) | | |
| Dynorphin A (1-6) | 0.12 ± 0.00* | 2 |
| (Tyr-Gly-Gly-Phe-Leu-Arg) | | |
| [Arg⁶, Phe⁷] Met-enkephalin | 0.11 ± 0.01* | 1 |
| (Tyr-Gly-Gly-Phe-Met-Arg-Phe) | | |
| [Arg⁶, Gly⁷, Leu⁸[ | 0.09 ± 0.01* | 1 |
| Met-enkephalin | | |
| (Tyr-Gly-Gly-Phe-Met-Arg- | | |
| Gly-Leu) | | |
| Dynorphin A (1-7) | 0.10 ± 0.02* | 1 |
| (Tyr-Gly-Gly-Phe-Leu-Arg-Arg) | | |
| Dynorphin B (1-9) | 0.08 ± 0.01* | 1 |
| (Tyr-Gly-Gly-Phe-Leu-Arg-Arg- | | |
| Gln-Phe) | | |
| Dynorphin A (1-13) | 0.07 ± 0.01*1 | |
| (Tyr-Gly-Gly-Phe-Leu-Arg-Arg- | | |
| IIe-Arg-Pro-Lys-Leu-Lys) | | |
| Naloxone (Opiate receptor | 8.38 ± 0.10 | 108 |
| antagonist) | | |
| Naltrexone (Opiate receptor | 8.22 ± 0.15 | 106 |
| antagonist) | | |

Uptake of [³H] deltorphin II (50 nM) was measured in Tat-ARPE-19 cells for 30 minutes in the presence of NaCl.
The final concentration of inhibitor was 1 mM.
*P < 0.05 compared with control.

TABLE 3
Substrate selectivity of the deltorphin II
transport system in control ARPE-19 cells.

| | [³H]Deltorphin II uptake | |
|---|---|---|
| Opioid peptide/antagonist | (pmol/mg protein/30 min) | (%) |
| Control | 1.08 ± 0.015 | 100 |
| Deltorphin II | 0.17 ± 0.006* | 16 |
| Met-enkephalin | 0.06 ± 0.004* | 5 |
| [Arg⁶, Phe⁷]Met-enkephalin | 0.05 ± 0.001* | 4 |
| [Arg⁶, Gly⁷, Leu⁸]Met-enkephalin | 0.04 ± 0.001* | 4 |
| Dynorphin A(1-6) | 0.06 ± 0.001* | 5 |
| Dynorphin A(1-7) | 0.05 ± 0.002* | 5 |
| Dynorphin A(1-13) | 0.04 ± 0.001* | 4 |
| Dynorphin B(1-9) | 0.06 ± 0.000* | 5 |
| Naltrexone | 1.12 ± 0.02 | 104 |
| Naloxone | 1.01 ± 0.00 | 94 |

ARPE-19 cells stably transfected with pcDNA vector alone were used as control cells. Uptake of [³H]deltorphin II (50 nM) was measured in these cells for 30 minutes in the presence of NaCl. Final concentration of opioid peptides/antagonists was 1 mM.
*P < 0.05 compared with control.

TABLE 4
Relative affinities of endogenous opioid peptides for the
deltorphin II transport system in Tat-ARPE-19 cells.

| Opioid peptide | IC₅₀ (mM) |
|---|---|
| Dynorphin A13 | 0.4 ± 0.1 |
| [Arg⁶, Gly⁷, Leu⁸]Met-enkephalin | 2.5 ± 0.4 |
| Dynorphin B(1-9) | 3.1 ± 0.6 |
| Dynorphin A(1-6) | 4.2 ± 0.6 |
| Dynorphin A(1-7) | 7.3 ± 1.9 |
| [Arg⁶, Phe⁷]Met-enkephalin | 9.1 ± 1.7 |
| Leu-enkephalin | 9.5 ± 1.8 |
| Met-enkephalinamide | 13.0 ± 2.7 |
| [des-Tyr¹]enkephalin | 31.9 ± 5.5 |
| Met-enkephalin | 33.2 ± 4.4 |
| Deltorphin II | 38.8 ± 4.6 |

IC₅₀ values (i.e., concentration of the peptide needed for 50% inhibition) were calculated from the dose-response curves in FIG. 2 for the inhibition of [³H]deltorphin II (25 nM) uptake by the peptides.

This example shows for the first time that human RPE cells express a Na⁺- and Cl⁻-coupled active transport system for a variety of opioid peptides consisting of 4-13 amino acids. Such a transport system has not been identified previously in any mammalian cell. A family of transport systems for bioactive peptides has been described in the blood/brain barrier and this family consists of at least four distinct subtypes, known as peptide transport systems 1-4 (PTS-1, PTS-2, PTS-3, and PTS-4) (Banks and Kastin, Am. J. Physiol., 1990;259:E1-E10; Banks et al., Peptides, 1987;8:899-903), but none of these transport systems exhibits the ion-dependence and substrate selectivity characteristics of the opioid peptide transport system described in this paper. Among the four subtypes of PTS family, PTS-1 is almost exclusively responsible for the transport of enkephalins (Banks et al., Am. J. Physiol., 1986;251:E477-E482). However, the transport system reported in the present study is not related to PTS-1, because there are marked differences between the two systems in terms of energy dependence and substrate specificity. PTS-1 is an energy-independent facilitative transport system (Banks and Kastin, Am. J. Physiol., 1990;259:E1-E10; Banks et al., Peptides, 1987;8:899-903; and Banks et al., Am. J. Physiol., 1986;251:E477-E482), whereas the transport system described in the present study is a Na⁺- and Cl⁻-coupled transport system. The opioid-related peptide Tyr-MIF-1 is widely used as a selective model substrate for PTS-1 (Banks et al., Peptides, 1987;8:899-903), but this peptide is not recognized by the opioid peptide transport system in RPE cells.

In addition, dynorphin A13 exhibits almost 10-fold higher affinity than the shorter dynorphins, which are made of only six to nine amino acids, for the opioid peptide transport system in RPE cells whereas, among the various dynorphins tested, dynorphin 1-8 shows the highest affinity for PTS-1 (Banks et al., Am. J. Physiol., 1986;251:E477-E482). These data show that none of the peptide transport systems, known to exist in the blood/brain barrier, represents the transport system described in the current study. Another transport system for the synthetic opioid peptide [D-penicillamine^{2,5}]enkephalin (DPDPE) has been described in the blood/brain barrier (Thomas et al., J. Pharmacol. Exp. Ther., 1997;280:1235-1240; Williams et al., J. Neurochem., 1996;66:1289-1299). But, unlike the opioid peptide transport system in RPE cells, this system does not recognize Leu-enkephalin as a substrate. The organic anion transporting polypeptides such as rat Oatp1 and Oatp2 and human OATP-A and OATP8 can transport Leu-enkephalin and deltorphin II as does the opioid peptide transport system in RPE cells, but these transport systems are Na⁺-independent (Gao et al., J. Pharmacol. Exp. Ther., 2000;294:73-79; Cattori et al., Pflugers Arch., 2001 ;443:188-195; and Kullak-Ublick et al., Gastroenterolgy, 2001; 120:525-533).

Furthermore, the non-peptide opioid receptor antagonist naloxone is a substrate for these organic anion transporting polypeptides (Gao et al., J. Pharmacol. Exp. Ther., 2000;294:73-79), whereas the opioid peptide transport system identified in RPE cells in the present study does not interact with this compound. PEPT1 and PEPT2, expressed in the mammalian intestine, kidney, brain, and lung, are peptide transport systems, but these transporters recognize only dipeptides and tripeptides as their substrates and exclude peptides larger than tripeptides (Leibach and Ganapathy, Annu. Rev. Nutr., 1996;16:99-119; Daniel, J. Membr. Biol., 1996;154:197-203; Inui and Terada, Pharmaceut. Biotechnol., 1999;12:269-288; and Ganapathy and Miyauchi, Am. Pharmaceut. Rev., 2003;6:14 -18). Moreover, PEPT1 and PEPT2 are Na⁺-independent and are driven by a transmembrane H⁺ gradient. Finally, an oligopeptide transport system (OPT1) has been described in Saccharomyces cerevisiae that can transport enkephalins, but this yeast transport system is H⁺-coupled and recognizes not only enkephalins but also the non-peptide opiate receptor antagonist naloxone (Hauser et al., J. Biol. Chem. 2000;275:3037-3041).

Thus, based on the ion-dependence and substrate selectivity characteristics, the opioid peptide transport system described in RPE cells in the present study is not identical with any of the previously identified transport systems in mammalian cells that can interact with opioid peptides. Since opioid peptides are involved in important biological functions in mammalian tissues, the newly identified ion-coupled active transport system, selective for a variety of endogenous opioid peptides, is likely to play a crucial role in the biology of these peptides.

As important as the discovery of this new opioid peptide-selective active transport system in mammalian cells is the finding that HIV-1 Tat up-regulates this transport system. This example identifies this transport system in RPE cells. Since endogenous opioid peptides function as neurotransmitters, it is speculated that the newly identified transport system may also be expressed in the brain to modulate the extracellular levels of these peptides. Endogenous opioid peptides include Leu-enkephalin, Met-enkephalin, and dynorphins, all of which are high-affinity substrates for the newly identified transport system. The plasma levels of enkephalin-like material in man are in the range of 0.1-1 nM (Ryder and Eng, J. Clin. Endocrinol. Metab., 1982;52:367-369) and the plasma half-lives for these peptides are in the range of 2-10 minutes (Hambrook et al., Nature, 1976;262:782-783; and Roda et al., (1983) “Stability of peripheral enkephalins.” In Degradation of Endogenous Opioid (Ehrenpreis, S. and Sicuteri, F., eds.), pp. 25-42, Raven Press, New York). These peptides are present in the central nervous system and gastrointestinal tract. The concentrations of enkephalins within the synapses of enkephalinergic neurons are likely to be several-fold higher than the plasma levels. It is currently believed that the half-lives of enkephalins in the plasma are determined primarily by peptidases in the circulation (Roda et al., (1986) “Control mechanisms in the enzyme hydrolysis of adrenal-released enkephalins.” In Enkephalins and Endorphins. Stress and the Immune System (Plotnikoff, N. P., Faith, R. E., Murgo, A. J. and Good, R. A., eds.), pp. 17-33. Plenum Press, New York).

The results of the present study suggest that the newly identified opioid transport system is also likely to play a significant role in the disposal of enkephalins from the circulation by mediating their entry into cells. These peptides serve as ligands for different classes of opiate receptors that are expressed on the plasma membrane of target cells (Chaturvedi et al., Biopolymers, 2000;55:334-346; and Kieffer, et al., Prog. Neurobiol., 2002;66:285-306). In addition to these plasma membrane receptors, mammalian cells express a nuclear receptor for enkephalins (Zagon et al., Brain Res. Rev., 2002;38:351-376). This receptor, known as opioid growth factor receptor (OGFr), plays a crucial role in cell proliferation and wound healing. The opioid peptide transport system might serve as an important determinant of not only the ligand concentration in the extracellular medium for interaction with the plasma membrane receptors, but also the ligand concentration in the intracellular medium for interaction with the nuclear receptor. Changes in the expression levels of this transport system are likely to lead to significant alterations in cellular signaling mediated by these receptors and consequently in the biological functions of opioid peptides. Therefore, the up-regulation of the transport system by HIV-1 Tat may have important clinical and pathological consequences in patients infected with HIV-1.

### Example 2

### Characterization of the Enkephalin/Endorphin Transport System in Human Neuronal Cell Line SK—N—SH

As shown in Example 1, studies with the human retinal pigment epithelial cell line ARPE-19 show that the enkephalin/endorphin transport system is Na⁺- and Cl⁻-dependent and is specific for enkephalins and endorphins. Since enkephalins and endorphins are relevant to pain perception and to further provide evidence in support of a role for the transport system in opioidergic neurotransmission, the human neuronal cell line SK—N—SH was tested for expression of the transport system. This example demonstrates that this transport system is expressed in neurons; that SK—N—SH cells express high levels of Na⁺/Cl⁻-coupled transport activity for opioid peptides. Unless otherwise specified, procedures are as detailed in Example 1.

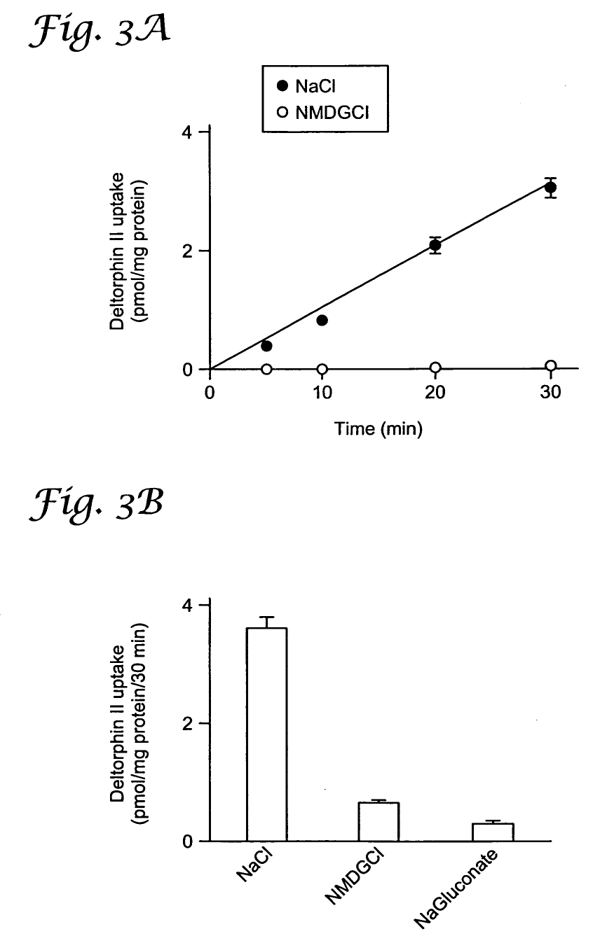

Both a time course and the ion-dependence of deltorphin II uptake in SK—N—SH cells were determined. The uptake of [³H]deltorphin II (25 nM) was measured in SK—N—SH cells first in the presence of NaCl for varying time periods (see FIG. 3A) and then under various ionic conditions with a 30 min incubation (see FIG. 3B). Thus, as shown in FIG. 3, SK—N—SH cells do express a deltorphin II uptake system as do the ARPE-19 cells. The uptake process is obligatorily dependent on the presence of Na⁺ as well as Cl⁻.

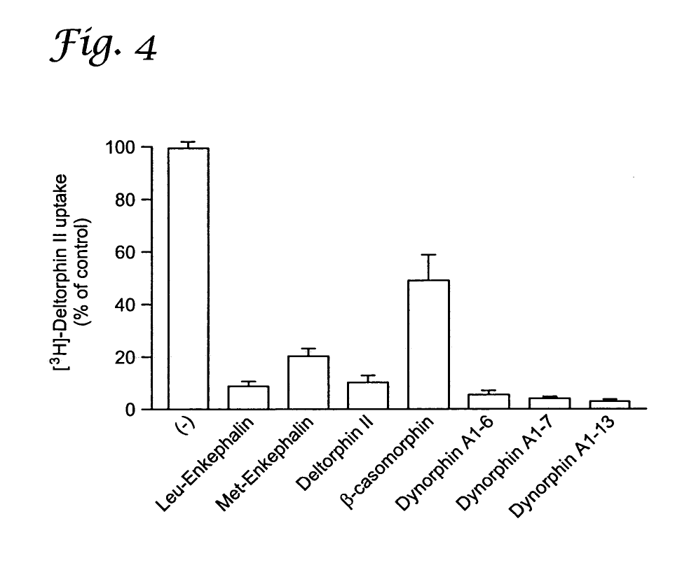

Next, the substrate selectivity of the deltorphin II uptake system in SK—N—SH cells was determined. The substrate selectivity of the uptake process was studied by assessing the effect of various enkephalins and dynorphins on the uptake of [³H]deltorphin II (25 nM) in SK—N—SH cells. The concentration of enkephalins and dynorphins was 1 mM. As shown in FIG. 4, the uptake of deltorphin II in SK—N—SH cells is inhibited by all of the enkephalins and dynorphins tested. Therefore, the substrate selectivity of the deltorphin II uptake system in SK—N—SH cells is the same as that of the transport system in ARPE-19 cells.

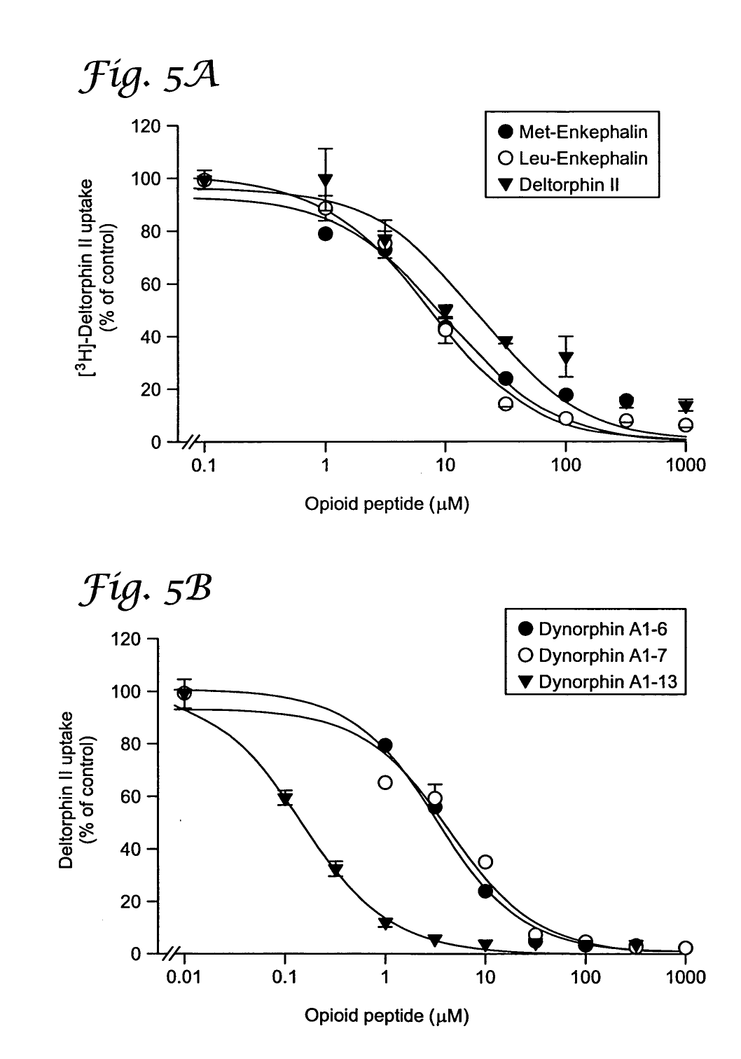

Finally, the relative affinity of various enkephalins and dynorphins for the transport system in SK—N—SH cells was determined. The affinities of various enkephalins and dynorphins for the transport system were determined by assessing the concentration-dependent inhibition of the uptake of [³H]deltorphin II (25 nM) in SK—N—SH cells. FIG. 5A shows results for Met-enkephalin, Leu-enkephalin, and deltorphin II. FIG. 5B shows results for Dynorphin A1-6, Dynorphin A1-7, and Dynorphin A1-13. The rank order of affinities for the various endogenous opioid peptides is in the following order: dynorphin A1-13 (0.15±0.01 μM) greater than dynorphin A1-6 (3.5±0.4 μM) greater than dynorphin A1-7 (4.6±1.1 μM) greater than Leu-enkephalin (7.8±1.2 μM) greater than Met-enkephalin (10.7±3.1 μM) greater than deltorphin II (19.5±7.8 μM). This rank order of affinities in SK—N—SH cells is similar to that in ARPE-19 cells. Thus, SK—N—SH cells express robust activity for deltorphin II uptake and the characteristics of the transport system in these cells are identical to those in control and Tat-expressing ARPE-19 cells.

### Example 3

### Identification of Specific Peptides as Positive Modulators of the Enkephalin/Endorphin Transport System in Human Neuronal Cell Line SK—N—SH

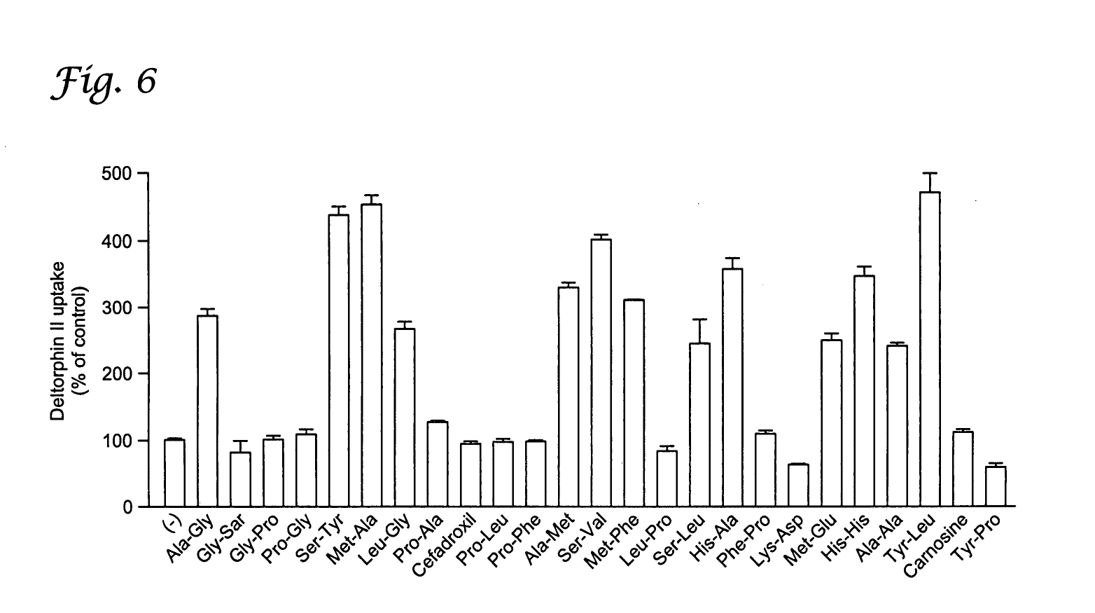

To the further characterize the opioid transport of the present invention, various specific peptides that act as positive modulators of the enkephalin/endorphin transport system in the human neuronal cell line SK—N—SH were identified. Unless otherwise specified, procedures are as detailed in Example 1. To determine the influence of various dipeptides on the enkephalin/endorphin transport system, the influence of various dipeptides on the enkephalin/endorphin transport system in SK—N—SH cells was studied by assessing their effects on the uptake of [³H]deltorphin II (25 nM). Uptake was measured in the presence of NaCl for 30 minutes. The concentration of the dipeptides was 1 mM. As shown in FIG. 6, many dipeptides show no ability to compete with deltorphin II for uptake, while several other dipeptides surprisingly show marked ability to stimulate the uptake of deltorphin II. The stimulation varies anywhere between 2 to 5-fold. This stimulatory effect of some of the dipeptides was unexpected.

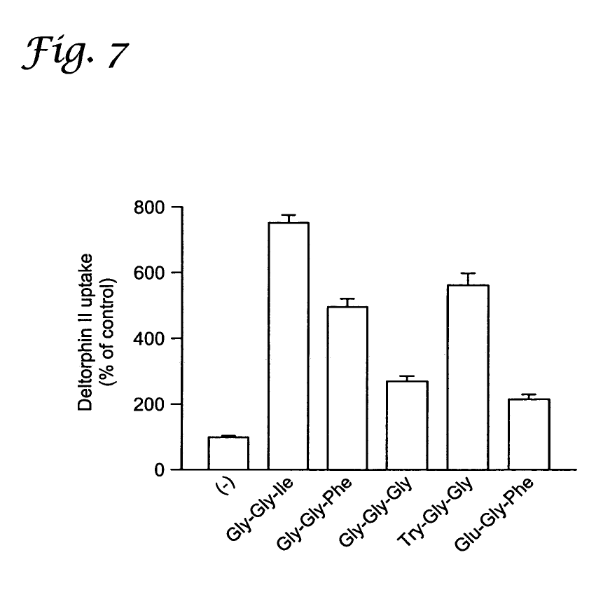

FIG. 7 shows the influence of various tripeptides on the enkephalin/endorphin transport system. The influence of various tripeptides on the enkephalin/endorphin transport system in SK—N—SH cells was studied by assessing their effects on the uptake of [³H]deltorphin II (25 nM). Uptake was measured in the presence of NaCl for 30 minutes. The concentration of the tripeptides was 1 mM. All five tripeptides tested (Gly-Gly-Ile; Gly-Gly-Phe; Gly-Gly-Gly; Try-Gly-Gly; and Glu-Gly-Phe) showed an ability to stimulate the uptake of deltorphin II. The stimulation varies in the range of 2 to 8-fold.

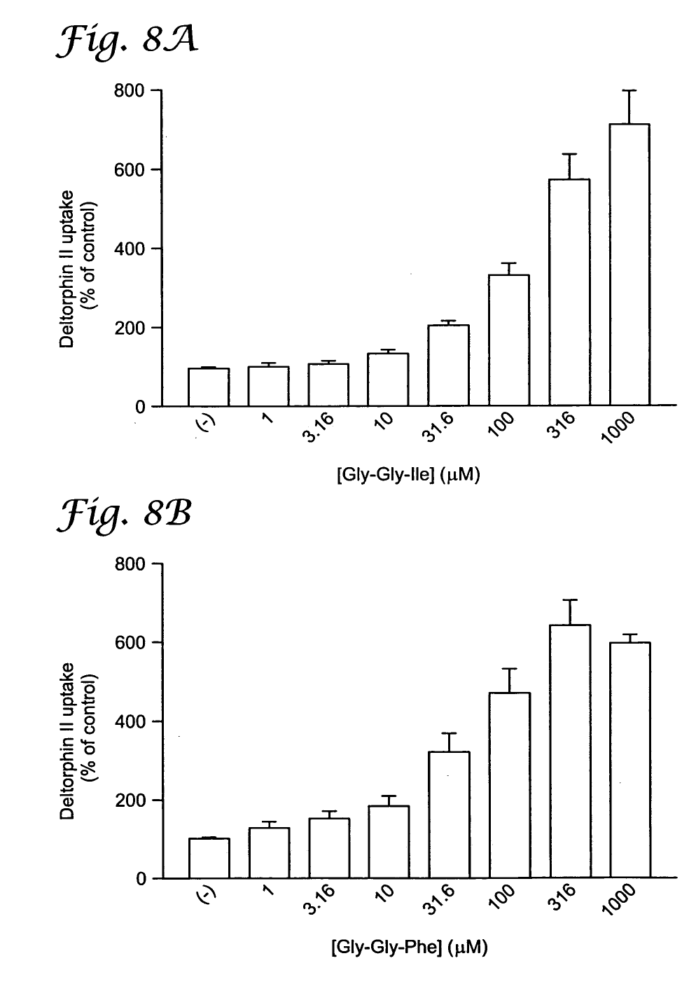

FIG. 8 shows a dose-response relationship for the stimulatory effect of Gly-Gly-Ile and Gly-Gly-Phe. The uptake of [³H]deltorphin II (25 nM) in SK—N—SH cells was measured in the presence of NaCl with a 30 minute incubation in the presence of varying concentrations of the two tripeptides. Significant stimulation was seen at the tripeptide concentrations as low as 10-30 μM.

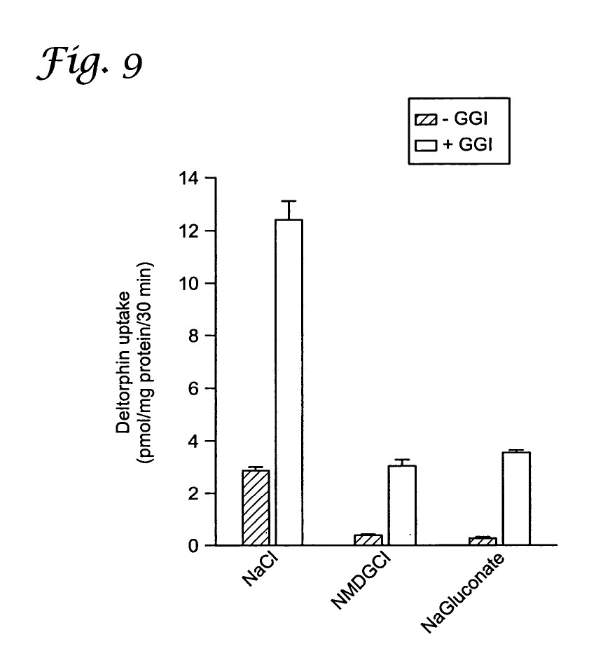

The ion-dependence of the enkephalin/endorphin transport system in the absence and presence of the stimulatory modifier Gly-Gly-Ile (GGI) is shown in FIG. 9. The uptake of [³H]deltorphin II (25 nM) in SK—N—SH cells was measured in the absence or presence of 1 mM Gly-Gly-Ile. The uptake buffer contained NaCl (i.e., the presence of both Na⁺ and Cl⁻), NMDG chloride (NMDGCl) (i.e., absence of Na⁺ but presence of Cl⁻), or sodium gluconate (Nagluconate) (i.e., presence of Na⁺ but absence of Cl⁻). The deltorphin II uptake was stimulated in the presence of Gly-Gly-Ile when measured in the presence of Na⁺ and Cl⁻. The uptake, in the absence or presence of Gly-Gly-Ile, is obligatorily dependent on the presence of Na⁺ as well as Cl⁻. Therefore, the ion-dependency of the transport system remains the same even in the presence of the stimulatory modifier Gly-Gly-Ile.

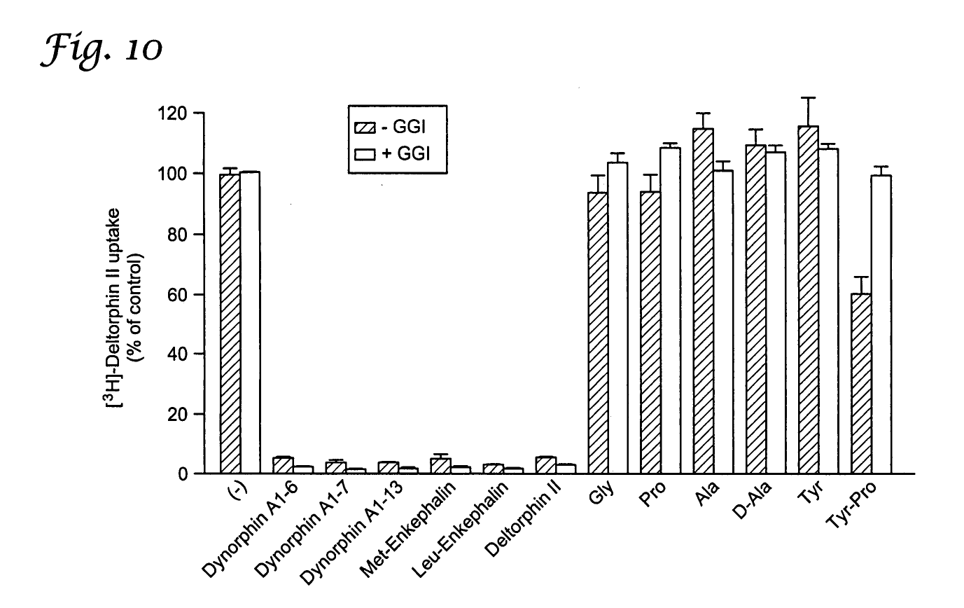

Substrate selectivity of the enkephalin/endorphin transport system in the absence and presence of the stimulatory modifier is shown in FIG. 10. The substrate selectivity of the transport system was studied in the absence or presence of 1 mM Gly-Gly-Ile (GGI) by assessing the influence of various enkephalins, endorphins, and amino acids on the uptake of [³H]deltorphin II (25 nM) in SK—N—SH cells. In the absence, as well as in the presence, of the stimulatory modifier Gly-Gly-Ile, the uptake system remains specific for Leu-enkephalin, Met-enkephalin, and dynorphins. Therefore, the substrate selectivity remains the same in the absence as well as in the presence of the stimulatory modifier.

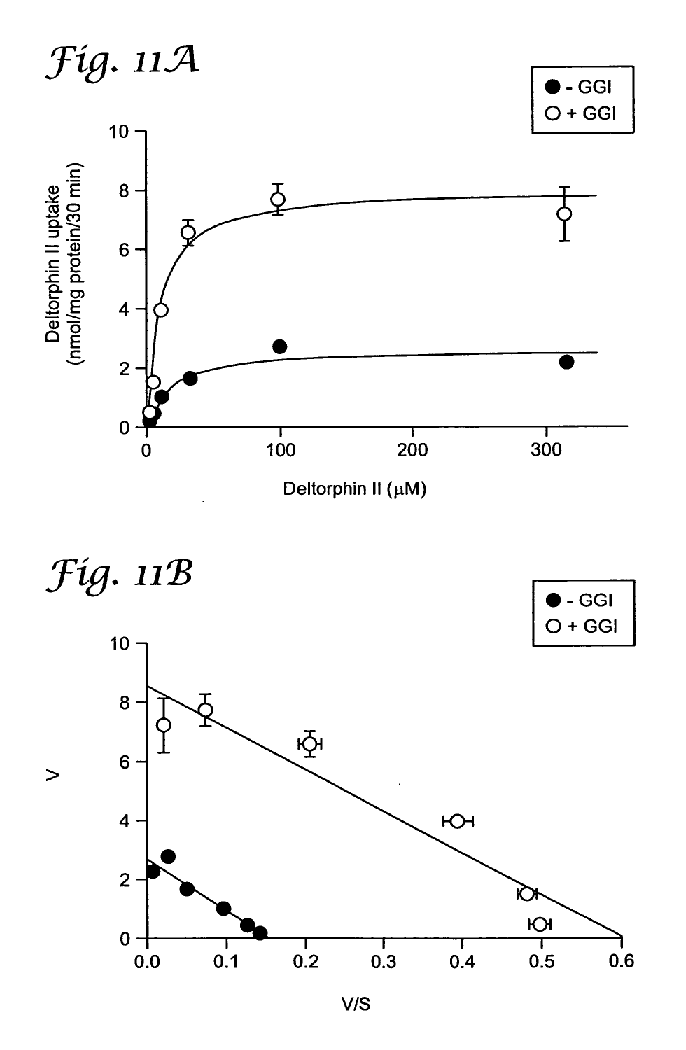

The influence of Gly-Gly-Ile on the kinetic parameters of enkephalin/endorphin transport system is shown in FIG. 11A-11B. The kinetics of deltorphin II uptake was studied in SK—N—SH cells in the absence or presence of 1 mM Gly-Gly-Ile. FIG. 11A shows deltorphin II concentration versus deltorphin II uptake. FIG. 11B shows deltorphin II uptake/deltorphin II concentration versus deltorphin II uptake. The stimulatory modifier Gly-Gly-Ile stimulates the enkephalin/endorphin transport system by increasing the maximal velocity of the system without affecting the affinity for its substrate.

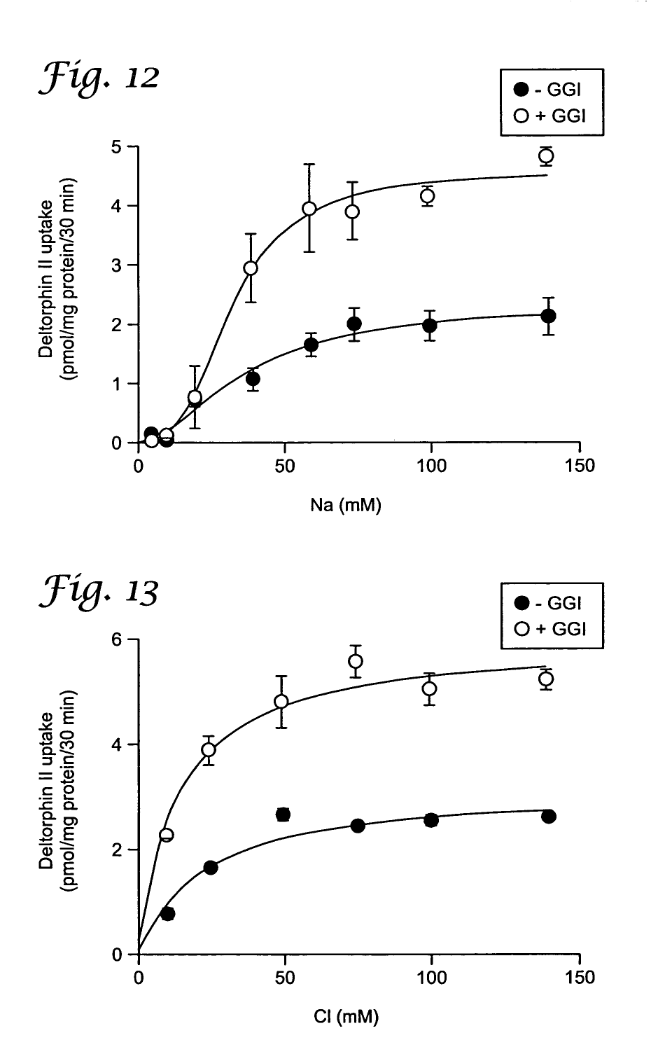

The influence of Gly-Gly-Ile on the Na⁺-activation kinetics of the enkephalin/endorphin transport system is shown in FIG. 12. The uptake of [³H]deltorphin II (25 nM) was measured in the absence or presence of Gly-Gly-Ile (1 mM) with varying concentrations of Na⁺ and with a fixed concentration of Cl⁻. More than one Na⁺ ion is involved in the transport process irrespective of whether or not the stimulatory modifier is present. The activation of the transport process by Na⁺ remains sigmoidal in the absence or presence of Gly-Gly-Ile.

The influence of Gly-Gly-Ile on the Cl⁻-activation kinetics of the enkephalin/endorphin transport system is shown in FIG. 13. The uptake of [³H]deltorphin II (25 nM) was measured in the absence or presence of Gly-Gly-Ile (1 mM) with varying concentrations of Cl⁻ and with a fixed concentration of Na⁺. Only one Cl⁻ ion is involved in the transport process irrespective of whether or not the stimulatory modifier is present. The activation of the transport process by Cl⁻ remains hyperbolic in the absence or presence of Gly-Gly-Ile.

### Example 4

### Identification of L-Lysine as a Negative Modulator of the Enkephalin/Endorphin Transport System in Human Neuronal Cell Line SK—N—SH

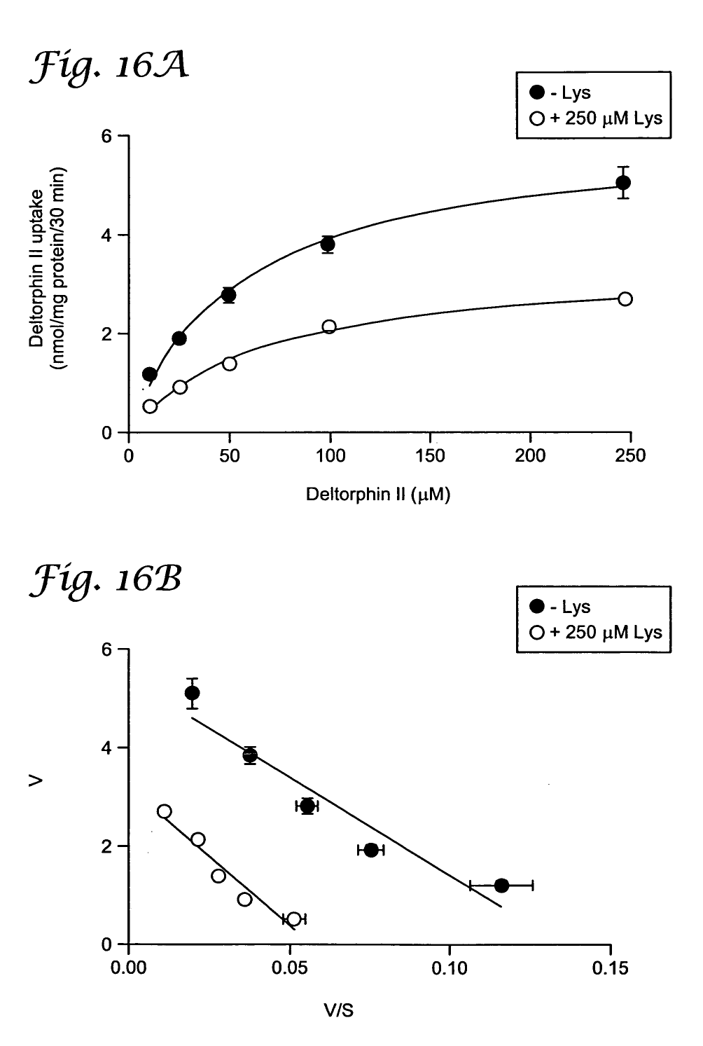

In this example, the selective inhibition of the opioid peptide transport system by L-lysine in SK—N—SH cells was determined. Unless otherwise specified, procedures are as described in more detail in Example 1. When the substrate specificity of the newly identified transport system was tested, it was surprisingly found that that L-lysine is a potent inhibitor of this transport system. Among the 15 different amino acids tested, L-lysine (1 mM) shows the greatest inhibition of deltorphin II (25 nM) uptake (FIG. 16). Under similar conditions, L-leucine, L-valine, D-alanine, D-tyrosine, and L-arginine also show significant inhibition, but the potency is much smaller than that seen with L-lysine. Various compounds structurally related to L-lysine were tested for the ability to inhibit deltorphin II uptake (deltorphin II concentration, 25 nM; inhibitor concentration, 1 mM). Only L-lysine and its methyl and ethyl esters are able to inhibit the uptake to a marked extent (Table 5). Interestingly, D-lysine has no effect, indicating the stereoselectivity for the inhibition. Dose-response studies have shown that the inhibition occurs with Ki values of 160±19 μM, 169±9 μM, and 154±34 μM for L-lysine, L-lysylmethyl ester, and L-lysylethyl ester, respectively.

Whether or not L-lysine is a transportable substrate for the opioid peptide transport system was tested. For this, uptake of L-lysine was studied and the influence of Na⁺ and opioid peptides assessed in SK—N—SH cells. These studies have shown that L-lysine uptake in these cells is not Na⁺-dependent and is insensitive to opioid peptides, indicating that L-lysine is not a transportable substrate for the opioid peptide transport system. This is supported by the kinetic analysis of the inhibition of deltorphin II uptake by L-lysine. The inhibition is non-competitive (FIG. 16B; Eadie-Hofstee plot: V, deltorphin II uptake in nmol/mg of protein/30 minutes; S, deltorphin II concentration in μM). L-Lysine decreased the maximal velocity of the transport system without affecting the affinity for deltorphin II. Thus, it appears that L-lysine is a blocker of the transport system and that the binding site for L-lysine does not overlap with the substrate-binding site on the transporter.

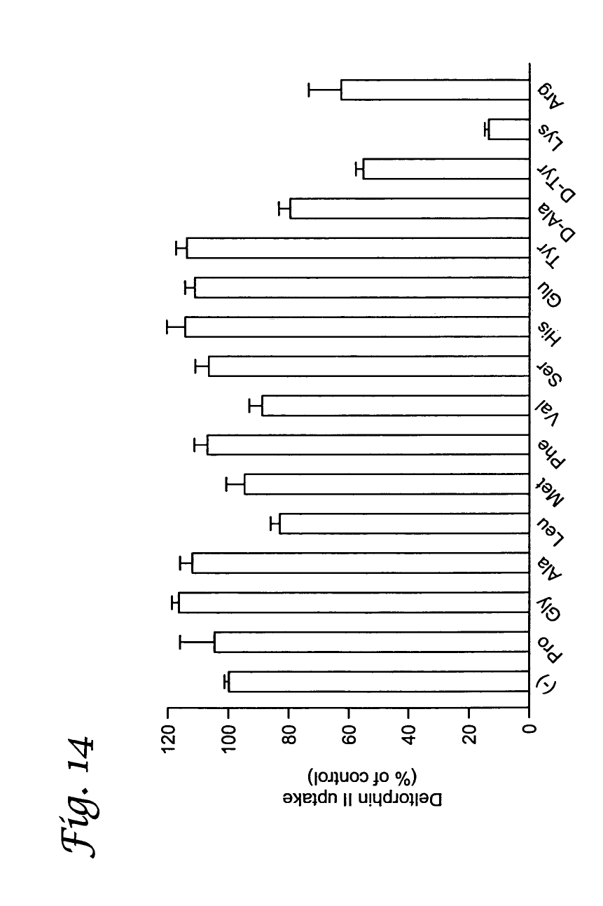

The influence of various amino acids on the enkephalin/endorphin transport system in SK—N—SH cells is shown in FIG. 14. The uptake of [³H]deltorphin II (25 nM) was measured in SK—N—SH cells in the presence of NaCl for 30 minutes in the absence or presence of various amino acids (1 mM). Most amino acids do not have any effect on the uptake process. However, D-tyrosine, L-Arginine, and L-Lysine show significant inhibition of uptake. L-Lysine is the most potent, causing 90% inhibition at a concentration of 1 mM.

Table 5, below, shows the influence of Lysine and structurally related compounds on the enkephalin/endorphin transport system in SK—N—SH cells. The uptake of [³H]deltorphin II (25 nM) was measured in SK—N—SH cells in the presence of NaCl for 30 minutes in the absence or presence of Lysine or structurally related compounds (1 mM). Only L-Lysine and its a-carboxy ester derivatives are the most potent inhibitors of the uptake process.

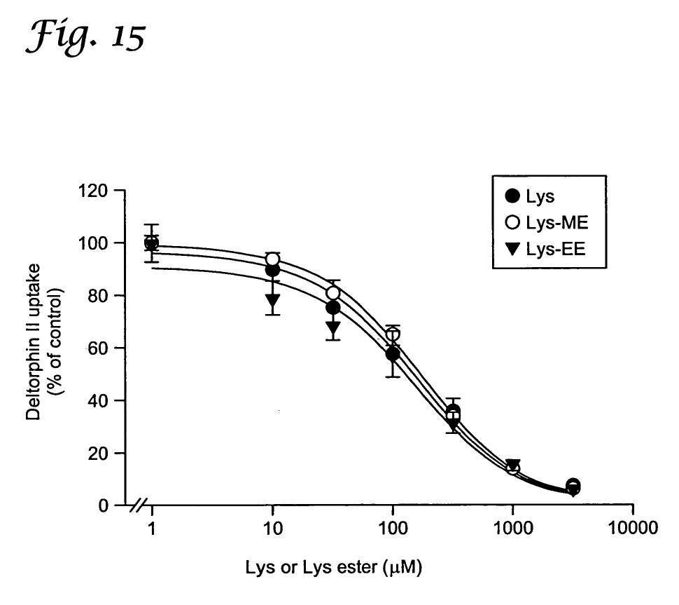

The dose-response relationship for the inhibition of the enkephalin/endorphin transport system by L-Lysine and its methyl and ethyl esters is shown in FIG. 15. The uptake of [³H]deltorphin II (25 nM) was measured in SK—N—SH cells in the presence of NaCl for 30 minutes in the absence or presence of increasing concentrations of L-Lysine or its esters. L-Lysine and its methyl and ethyl esters are equally potent as inhibitors of deltorphin II uptake in SK—N—SH cells with an approximate IC₅₀ value of 150 μM.

The influence of L-Lysine on the kinetics of the enkephalin/endorphin transport system is shown in FIG. 16A-16B. The kinetics of the enkephalin/endorphin transport system was studied in the absence or presence of 250 μM L-Lysine by using deltorphin II as the substrate for the transport system. FIG. 16A shows deltorphin II concentration versus deltorphin II uptake. FIG. 16B shows deltorphin II uptake/deltorphin II concentration versus deltorphin II uptake. L-Lysine inhibits the transport system by decreasing the maximal velocity without affecting the substrate affinity.

TABLE 5

| | Deltorphin II uptake | |
|---|---|---|
| | (pmol/30 min/mg protein) | % inhibition |
| (−) | 3.00 ± 0.19 | 100 |
| L-Lys | 0.26 ± 0.01 | 9 |
| L-Arg | 1.46 ± 0.12 | 49 |
| GABA | 2.62 ± 0.13 | 87 |
| L-Ornitine | 1.87 ± 0.08 | 62 |
| L-Carnitine | 2.34 ± 0.12 | 78 |
| L-Citrulline | 2.18 ± 0.06 | 73 |
| D-Lys | 2.60 ± 0.31 | 87 |
| Trimethyl-L-Lys | 2.39 ± 0.11 | 80 |
| L-Lys-amide | 2.92 ± 0.09 | 97 |
| L-Lys-methyl ester | 0.28 ± 0.01 | 9 |
| Lys-ethyl ester | 0.55 ± 0.05 | 18 |
| α-N-acetyl-L-Lys | 2.82 ± 0.08 | 94 |
| ε-N-acetyl-L-Lys | 2.38 ± 0.13 | 79 |
| α-N-acetyl-L-Lys-methyl | 2.37 ± 0.20 | 79 |
| ester | | |
| δ-amino-levulinic acid | 2.68 ± 0.18 | 89 |
| L-NIL | 2.40 ± 0.15 | 80 |
| 1,5-diaminopentane | 3.24 ± 0.16 | 108 |
| Hexamethylenediamine | 2.83 ± 0.13 | 94 |
| 6-amino hexanoic acid | 2.92 ± 0.18 | 97 |

### Example 5

### The Interaction of L-Lysine and its Derivatives with the Opioid Peptide Transport System Using Primary Neuronal Cell Cultures

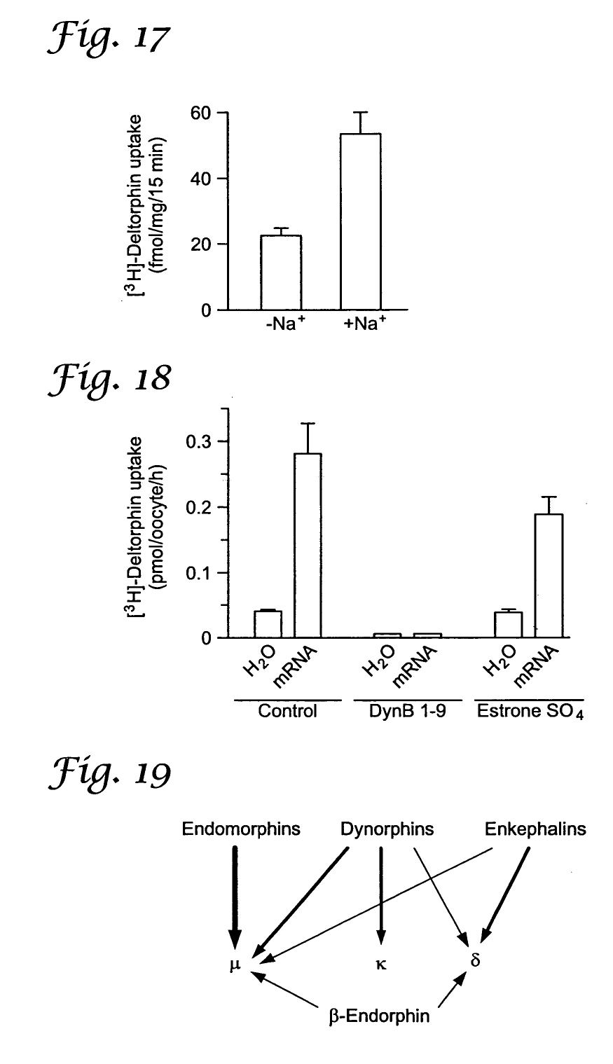

Examples 1-4 characterize the novel opioid transport system using the cultured cell lines ARPE-19 and SK—N—SH. To demonstrate the existence of the transport system be demonstrated in normal brain, neurons derived from striatum were tested for Na⁺-dependent deltorphin II uptake, as this region of the brain is known to contain high levels of opioid peptides (Saria et al., (1997) Neurosci. Lett. 234: 27-30). As shown in FIG. 17, the uptake of deltorphin II (25 nM) in these neuronal cultures is stimulated 2-fold in the presence of Na⁺. Specifically, the uptake of deltorphin II is 22.5±2.3 fmol/mg of protein/15 minutes in the absence of Na⁺ and 53.5±6.4 fmol/mg of protein/15 minutes in the presence of Na⁺. Hypothalamus, brain stem, and spinal cord also contain high levels of opioid peptides and it is likely that the opioid peptide transport system is also expressed in these regions as well. The expression of the novel opioid transport system in primary neuronal cultures from rat brain indicates that the transport system is indeed expressed in normal brain. With this example, the interaction of L-lysine with the transport system in primary cultures of neuronal cells will be further studied. These studies will be carried out with rat striatal neuronal cells. The opioid peptide transport system will be characterized in primary cultures of rat striatal neurons in terms of critical features such as Na⁺/Cl⁻-dependence and substrate specificity and to investigate in detail the interaction of the transport system with L-lysine. Striatal neurons have been chosen because these neurons show enriched expression of opioid peptides and therefore these neurons are most likely to express the opioid peptide transport system.

Procedures used for establishing the primary culture of striatal neurons will be similar to those described previously by Prasad and Amara (Prasad and Amara, (2001) J. Neurosci. 21: 7561-7567). Timed pregnant rats (14-day gestation) will be obtained from Charles River Laboratories (Wilmington, Mass.) and maintained in the vivarium on the Medical College of Georgia campus. Rat pups (2-4 days old) will be anesthetized by intraperitoneal injection of ketamine HCl (3 mg/pup) and striatal tissue including globus pallidus will be dissected into sterile Hank's balanced salt solution (HBSS). Tissue will be washed thrice in HBSS and then incubated in a dissociation medium containing 20 Units/ml activated papain, at 34-36° C. under continuous oxygenation for 2 hours. Tissue will then be dissociated with fire-polished Pasteur pipette in minimum essential medium. Dissociated cells will be plated in 48-well tissue culture dishes that were previously coated with 100 μg/ml polylysine and 5 μg/ml laminin at a density of approximately 150,000 cells per well. A medium comprising of 50% minimum essential medium, 40% Ham's-F12 medium, 10% heat-inactivated horse serum, 0.45% D-glucose, 5 pg/ml insulin and 0.1 mg/ml apotransferrin will be used to maintain the neuronal cultures. Cultures will be maintained for two weeks in vitro to allow for the development of neuronal processes and optimal transporter expression. Uptake measurements on two-week old cultures will be performed as described below. The culture medium will be removed by aspiration and uptake buffer containing [³H]deltorphin II will be added to the cells to initiate uptake. After incubation at 37° C. for a desired time, uptake will be terminated by the removal of the medium and washing of the cells with ice-cold uptake buffer. The cells will then be dissolved in 1% sodium dodecyl sulfate in 0.2 N NaOH and used for measurement of radioactivity. The uptake buffer in most experiments is 25 mM Hepes/Tris (pH 7.5), containing 140 mM NaCl, 5.4 mM KCl, 1.8 mM CaCl₂, 0.8 mM MgSO₄, and 5 mM glucose. When the influence of Na⁺ on the uptake process is investigated, the concentration of NaCl in the uptake buffer is adjusted, as desired, by isoosmotically replacing NaCl with N-methyl-D-glucamine chloride. To assess the influence of Cl⁻ on the uptake process, the composition of the uptake medium is modified by replacing KCl and CaCl₂ with equimolar concentrations of potassium gluconate and calcium gluconate and the concentration of NaCl is adjusted, as desired, by isoosmotically replacing NaCl with sodium gluconate. Saturation kinetics will be analyzed by fitting the data to Michaelis-Menten equation. The Michaelis-Menten constant, Kt, will be calculated by non-linear regression analysis and then confirmed by linear regression. Na⁺- and Cl⁻-activation kinetics will be analyzed by fitting the data to the Hill equation and the Hill coefficients for Na⁺ and Cl⁻ (h; the number of Na⁺ and Cl⁻ ions involved in the activation process) and the K0.5 values (the concentration of Na⁺ and Cl⁻ needed for half-maximal activation) will be determined from the analysis. Experiments will be repeated three times, each experiment done in duplicate. The interaction of the transport system with L-lysine and its derivatives will be carried out as previously described for SK—N—SH cells. These studies would include dose-response relationship, specificity, and inhibition kinetics. The purpose of these studies is to establish that the opioid peptide transport system in primary neuronal cultures exhibits functional features, in particular with respect to inhibition by L-lysine, that are similar to those found in SK—N—SH cells.

To optimize the expression of the opioid peptide transport activity cells will be cultured in the presence of various nerve growth factors determine the optimal culture conditions. Certain tripeptides, for example, Gly-Gly-Ile, stimulate deltorphin II uptake by 6- to 8-fold in SK—N—SH cells. It will be determined whether this phenomenon observed in the human cell line also occurs in rat neuronal cultures. If so, transport activity can be enhanced for study either by changes in culture conditions or by the addition of peptides such as Gly-Gly-Ile.

### Example 6

### Cloning the Opioid Peptide Transporter by Functional Expression in Xenopus laevis oocytes

Since the newly identified opioid peptide transport system is coupled to Na⁺ and Cl⁻, experimental efforts were made to determine if the opioid transporter responsible of the present invention belonged to the SLC6 gene family. This family contains all of the previously known Na⁺/Cl⁻-coupled transporters including all of the known neurotransmitter transporters (Chen et al., (2004) Pflugers Arch.-Eur. J. Physiol. 447: 519-531; and Broer et al., (2004) J. Biol. Chem. 279: 24467-24476). The human genome project has shown that this family consists of 20 members. But, the functional identity of only four members in this gene family remains unknown. These four putative transporters are XT2, XT3, NTT4, and V7-3. Each of these four were cloned and tested for their ability to transport deltorphin II. But, none of these transporter clones showed deltorphin II uptake activity and it is possible that the Na⁺/Cl⁻-coupled opioid peptide transport system is a member of some gene family other than SLC6. Next, attempts were made to clone the transport system by functional complementation in S. cerevisiae. This approach has been successfully used to clone the yeast enkephalin transport system (Hauser et al., (2000) J. Biol. Chem. 275: 3037-3041). Attempts to clone the opioid peptide transport system from SK—N—SH cells using this approach have to date been unsuccessful.

Therefore, functional expression of the deltorphin II transport system in X. laevis oocytes will be used to clone this transport system. The success of this approach relies heavily on the selection of the source mRNA that will induce deltorphin II uptake in oocytes to a significant extent. Therefore, a variety of cell lines were screened for deltorphin II uptake activity. And it was found that rMC-1 cells, a rat Müller cell line, express robust activity of this transport system. rMC-1 cells are the retinal glial cells and opioidergic neurotransmission is known to have biological functions in the retina (Su et al., (1986) Cell. Mol. Neurobiol. 6:331-347; Abe et al., (1994) Peptides 15: 49-54; and Seltner et al., (1997) Vis. Neurosci. 14: 801-809). Poly(A)+ mRNA was prepared from this cell line for functional expression studies in X. laevis oocytes. Injection of mRNA into oocytes induced deltorphin II (125 nM) uptake about 7-fold (see FIG. 18). This uptake was completely inhibitable by dynorphin B 1-9. In contrast, estrone-3-sulfate, a substrate for OATP-A, showed only a slight inhibition. These data show that the deltorphin II uptake inducible by rMC-1 mRNA represents the newly identified opioid peptide transport activity.

When information on the molecular nature of a given transport system is not available, the technique of functional expression cloning can be used to clone the transporter responsible for the transport activity. This approach has been used to successfully clone the intestinal peptide transporter successfully (Fei et al., (1994) Nature 368: 563-566). For this technique to succeed, an mRNA source needs to be selected that gives a robust signal for deltorphin II uptake in X. laevis oocytes when injected with the mRNA. Experiments discussed above indicate that the rat Müller cell line rMC-1 is the best for this purpose. Injection of poly(A)+ mRNA from this cell line induces deltorphin II uptake in oocytes by about 8-fold. This mRNA will be used to clone the transport system.

Size-fractionation of rMC-1 mRNA and construction of cDNA library. AS shown in FIG. 18, microinjection of rMC-1 poly(A)+ RNA into Xenopus oocytes results in functional expression of the opioid peptide transport system. In order to enrich the transporter mRNA prior to the construction of the cDNA library, mRNA will be subject to size-fractionation by centrifugation through a sucrose density gradient as described by Palacin et al. (Palacin et al., (1990) J. Biol. Chem. 265: 7142-7144). The oocyte expression system will be used to determine the relative enrichment of the transporter message in fractionated RNA pools by assaying for deltorphin II uptake.

Fractionation of RNA by this technique occurs based on the size of the RNA. The RNA fractions will be injected individually into oocytes and the induction of opioid peptide uptake will be monitored. The RNA fraction that shows maximal induction of deltorphin II uptake activity will then be used for construction of the cDNA library. SuperScript Plasmid System (Gibco-BRL) may be used for this purpose. The mRNA fraction will be reverse transcribed and the resultant cDNAs ligated into the Not I/Sal I-digested pSPORT 1. These constructs will be electroporated into ElectroMax DH10B cells. The cDNA inserts in these constructs are under control of T7 promoter and therefore are suitable for cRNA synthesis using T7 RNA polymerase following linearization of the plasmids.

Screening of the cDNA library for the opioid peptide transporter. For the first round of screening, the cDNA library will be divided into multiple pools, each pool consisting of approximately 500 clones. Plasmid DNA will be isolated from each pool, gene cleaned and transcribed using T7 RNA polymerase in the presence of a cap analog after linearization of the plasmid by Not I digestion. The resulting cRNA from each pool will be microinjected into oocytes and deltorphin II uptake will be monitored after 3-4 days. Uninjected oocytes will be used as controls. The plasmid pool that shows maximal induction of the opioid peptide transport activity will then be further divided into multiple pools containing lesser number of clones and the above-described steps will be repeated until a single clone that exhibits the opioid peptide transport activity is obtained. Once this is achieved, the cDNA insert will be sequenced and the primary structure of the predicted protein will be established.

Cloning of the human opioidpeptide transporter. Once the functional and molecular identity of the opioid peptide transport system from rat Müller cells has been established, the transporter cDNA will be used as a probe to clone the human transporter. A cDNA library will be constructed for SK—N—SH cells and screened to isolate the human ortholog. It is possible that the transport system might consist of more than one subunit, as some amino acid transporters do exist as heterodimers. If this is the case for the opioid peptide transporter, transport activity signal in oocytes will be lost during screening of the cDNA library because the mRNA species coding for the two different subunits are not expected to stay together in the same pool of the library. If this happens, it will be apparent that the transport system is not the product of a single gene. If that is the case, changes will be made to the screening procedure. This will involve mixing two different pools of the library for the detection of the transport signal and screening both pools until single clones obtained from both pools induce the transport signal when co-expressed.

### Example 7

### The Interaction of L-Lysine and its Derivatives with the Cloned Transporter in Heterologous Expression Systems

Examples 1-6 show that the opioid peptide transport system in SK—N—SH cells and in rMC-1 cells is inhibited by L-lysine. Once clones responsible for the transport activity in these cells are isolated, transport function of the cloned transporters will be further characterized, with special emphasis on the interaction of L-lysine and its derivatives with the cloned transporter in heterologous expression systems. These studies will provide essential corroborative evidence in support of specific interaction of L-lysine with the opioid peptide transport system.

The cloned transporters will be expressed functionally in two different heterologous expression systems, a vaccinia virus expression system in mammalian cells and a X. laevis oocyte expression system. Radiolabeled deltorphin II will be used for monitoring the transport function of the expressed transporter in mammalian cells. A detailed analysis of the functional characteristics of the cloned rat and human transporters will be carried out using the same approach employed for SK—N—SH cells and rMC-1 cells, as detailed in Example 1-6. The human retinal pigment epithelial cell line HRPE will be used with the vaccinia virus expression system in analyzing the functional characteristics of cloned transporters (Hatanaka etal., (2004) J. Pharmacol. Exp. Ther. 308: 1135-1147; Inoue et al., (2004) Biochem. J. 378: 949-957; Miyauchi et al., (2004) J. Biol. Chem. 279: 13293-13296; and Gopal etal., (2004) J Biol Chem. 279(43):44522-32). As these cells express very low levels of deltorphin II uptake activity constitutively, these cells are ideal for heterologous expression of the cloned transporters. The experiments will include Na⁺-activation kinetics, Cl⁻-activation kinetics, substrate specificity, and saturation kinetics. Then, the interaction with L-lysine and its structural analogs will be analysed. For the X. laevis oocyte expression system, an electrophysiological approach will be used to monitor the transport function of the cloned transporters.

Since the transport function is Na⁺- and Cl⁻-coupled, it is highly likely that the transport process is electrogenic. Therefore, transport function can likely be monitored by substrate-induced inward currents using the two-microelectrode voltage-clamp technique. This approach has been successfully used to characterize several transporters (See, Hatanaka etal., (2004) J. Pharmacol. Exp. Ther. 308: 1135-1147; Inoue et al., (2004) Biochem. J. 378: 949-957; Miyauchi et al., (2004) J. Biol. Chem. 279: 13293-13296; and Gopal etal., (2004) J Biol Chem. 279(43):44522-32). This approach can be used not only for the analysis of functional characteristics but also for the investigation of the interaction of the transporters with L-lysine and its analogs. Studies with SK—N—SH cells and rMC-1 cells have shown that L-lysine is not a transportable substrate but a blocker for the opioid peptide transport system. This can be investigated using the electrophysiological approach by analyzing the blockade of opioid peptide-induced currents by L-lysine and its analogs.

### Example 8

### L-Lysine as an Analgesic and Antidiarrheal Agent in Rat

The analgesic and antidiarrheal actions of L-lysine will be evaluated in intact animals. Two different nociceptive tests will be used to evaluate the analgesic effect of the amino acid L-lysine in rats. The antidiarrheal potency of L-lysine will be evaluated using an experimental design in which diarrhea is induced in rats by a combination of constraint stress and 5-hydroxytryptophan.

There are several opiate-sensitive noxious tests (Nieto et al., (2001) Neuropharmacology 41: 496-506). Two such tests will be employed to assess the analgesic potential of L-lysine, namely the hot plate test and the tail flick test. These tests are standard procedures for the assessment of nociceptive function in small animals such as rats. To assay for the antidiarrheal effect of L-lysine, the method described by Smriga and Torii will be used in which diarrhea is induced by a combination of restraint stress and 5-hydroxytryptophan (Smriga and Torii (2003) Proc. Natl. Acad. Sci. (USA) 100: 15370-15375). In each test, the influence of L-lysine will be assessed by administering the amino acid as an oral infusion. All these tests will be carried out in rats.

Hot plate test. This test assesses the supraspinal nociception. Rats will be divided into three groups, with 20 rats per group. The control group of rats will be orally infused with water (5 ml) and the two experimental groups of rats (for two different doses of L-lysine) will be orally infused with L-lysine at two different doses (0.5 g/kg body weight or 1 g/kg body weight in 5 ml water). The rats will be subjected to hot plate test one hour after the oral infusion. Individual rats will be placed in the glass-enclosed section of a Hot-Plate Analgesia Meter (Accuscan Instruments, Inc., Columbus, Ohio). The temperature of the heating surface will be elevated by 3° C. per min from a beginning temperature of 42° C. to a maximum temperature of 49° C. The time elapsed (latency) before the rat lifts and/or licks a hind paw or jumps will be recorded as a measure of nociception. Each rat will be given 3 trials separated by a 30-minute (minimum) intertrial interval.

Tail flick test. This test assesses the spinal nociception. Rats will be divided into three groups, with 20 rats per group. The control group of rats will be orally infused with water (5 ml) and the two experimental groups of rats (for two different doses of L-lysine) will be orally infused with L-lysine at two different doses (0.5 g/kg body weight or 1 g/kg body weight in 5 ml water). The rats will be subjected to tail flick test one hour after the oral infusion. Testing will be conducted with the Tail Flick Analgesia Unit (San Diego Instruments, San Diego, Calif.) which measures nociception as indicated by a “tail flick” response to heating a small area of the tail of the rat. The animal's tail will be placed over a window on the platform and a foot switch will activate an intense light beam that will heat the tail at a reliable, reproducible rate. When the animal senses a sufficient level of discomfort, it flicks its tail, automatically stopping the timer. Thus, the time for the animal to move (flick) its tail away from the heat will be recorded as a measure of nociception. The reaction time from activation of the light beam to the tail flick is automatically presented on a digital display (timer resolution, 0.1 sec). Each rat will be given 3 trials separated by a 30-minute (minimum) intertrial interval.

Test for antidiarrheal potency. This test will be applied to fasting rats in which diarrhea is induced by a combination of restraint stress and 5-hydroxytryptophan (Smriga and Torii (2003) Proc. Natl. Acad. Sci. (USA) 100: 15370-15375). Rats will be divided into four groups, with 20 rats per group. Rats in all groups will be fasted for 24 hours. The rats in Group 1 and Group 2 will be orally infused with water (5 ml) and the rats in Group 3 and Group 4 will be orally infused with L-lysine (1 g/kg body weight in 5 ml water). One hour following oral infusion, the rats in Group 2 and Group 4 will be slightly anesthetized with isoflurane and their forepaws, upper forelimbs and thoracic trunks will be wrapped with adhesive tape. Then, the rats will be injected subcutaneously (s.c.) with 5-hydroxytryptophan (10 mg/kg body weight in 0.5 ml water). The rats in Group 1 and Group 3 will not be subjected to wrap restraint and 5-hydroxytryptophan injection. The incidents of diarrhea will be monitored for I hour from the time of s.c. injection by an experimenter blinded to treatments. If there is excretion of loose stools and no formed feces, the rat will be scored as having diarrhea. The incidence of diarrhea will be evaluated as the number of rats with loose stool per 20 rats in each group. This experimental design will allow us to determine the incidence of diarrhea induced by the combination of wrap restraint and 5-hydroxytryptophan administration in control rats (Group 2 versus Group 1) and also to evaluate the effect of L-lysine on the incidence of diarrhea induced by stress and 5-hydroxytryptophan (Group 4 versus Group 2). The influence of L-lysine alone on the incidence of diarrhea in control rats (Group 3 versus Group I) can also be assessed. The efficacy of L-lysine as an antidiarrheal agent can be evaluated by the difference in the incidence of diarrhea between Group 2 versus Group 1 and Group 4 versus Group 3.

Statistical analysis. In each test, treatment differences will be analyzed by two-way ANOVA followed by Newman-Keuls' test. When data from an experimental group are compared with the data from the corresponding control group, Dunnett's t-test will be used.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of identifying an agent that modulates the transmembrane transport of an endogenous opioid peptide by an endogenous opioid peptide transport system, the method comprising:
contacting a cell expressing an endogenous opioid peptide transport system with an agent, wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile and/or Gly-Gly-Phe;
and combinations thereof; and
determining the transmembrane transport of the endogenous opioid peptide by the endogenous opioid peptide transport system;
wherein a modulation in the transmembrane transport of the endogenous opioid peptide when the cell is contacted with the agent indicates the agent modulates the transmembrane transport of an opioid peptide by an endogenous opioid peptide transport system.

2. The method of claim 1, wherein the endogenous opioid peptide transport system exhibits an upregulation of the transport of endogenous opioid peptide by the Tat protein encoded by the human immunodeficiency virus type I.

3. The method of claim 1, wherein transport of an endogenous opioid peptide is coupled to a sodium gradient.

4. The method of claim 1, wherein transport of an endogenous opioid peptide is coupled to a chloride gradient.

5. The method of claim 1, wherein transport of an endogenous opioid peptide is coupled to a sodium gradient and a chloride gradient.

6. The method of claim 1, wherein transport of an endogenous opioid peptide is inhibited by L-lysine.

7. The method of claim 1, wherein transport of an endogenous opioid peptide is stimulated by the tripeptides Gly-Gly-Ile and/or Gly-Gly-Phe.

8. The method of claim 1, wherein the endogenous opioid peptide transport system exhibits upregulation of the transport of endogenous opioid peptide by the Tat protein encoded by the human immunodeficiency virus type I, transport of an endogenous opioid peptide is coupled to a sodium gradient, transport of an endogenous opioid peptide is coupled to a chloride gradient, inhibition of the transport of endogenous opioid peptide by L-lysine, and stimulation of the transport of endogenous opioid peptide by the tripeptides Gly-Gly-Ile and/or Gly-Gly-Phe.

9. A method of modulating the activity of an opioid, the method comprising administering an agent that modulates the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile and/or Gly-Gly-Phe;
and combinations thereof.

10. The method of claim 9, wherein the agent is selected from the group consisting of Gly-Gly-Ile, Gly-Gly-Phe, Gly-Gly-Gly, Try-Gly-Gly, Glu-Gly-Phe, L-lysine, L-valine, D-alanine, D-tyrosine, L-arginine, an analog or structural derivative, and combinations thereof.

11. The method of claim 9, wherein modulating is an increase in the transport of an endogenous opioid peptide.

12. The method of claim 11, wherein the agent is a tripeptide selected from the group consisting of Gly-Gly-Ile, Gly-Gly-Phe, Gly-Gly-Gly, Try-Gly-Gly, Glu-Gly-Phe, an analog or structural derivative thereof, and combinations thereof.

13. The method of claim 9, wherein modulation is an inhibition of the transport of an endogenous opioid peptide.

14. The method of claim 13, wherein the agent is selected from the group consisting of L-lysine, L-valine, D-alanine, D-tyrosine, L-arginine, an analog or structural derivative thereof, and combinations thereof.

15. The method of claim 14, wherein the agent is L-lysine or an analog or structural derivative thereof.

16. A method of treating pain, the method comprising administering an effective amount of an agent that inhibits the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile and/or Gly-Gly-Phe;
and combinations thereof.

17. The method of claim 16, wherein the agent is selected from the group consisting of L-lysine, L-valine, D-alanine, D-tyrosine, L-arginine, an analog or structural derivative thereof, and combinations thereof.

18. The method of claim 16, wherein the agent is L-lysine or an analog or structural derivative thereof.

19. A method of reducing the amount of narcotic needed for effective pain management, the method comprising administering an effective amount of an agent that inhibits the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine, stimulation of the transport of endogenous opioid peptide by the tripeptides Gly-Gly-Ile and/or Gly-Gly-Phe;
and combinations thereof.

20. The method of claim 19, wherein the likelihood of the development of addiction is reduced.

21. The method of claim 19, wherein the agent is selected from the group consisting of L-lysine, L-valine, D-alanine, D-tyrosine, L-arginine, an analog or structural derivative thereof, and combinations thereof.

22. The method of claim 19, wherein the agent is L-lysine or an analog or structural derivative thereof.

23. A method of decreasing the motility of the intestine, the method comprising administering an effective amount of an agent that inhibits the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine, stimulation of the transport of endogenous opioid peptide by the tripeptides Gly-Gly-Ile and/or Gly-Gly-Phe;
and combinations thereof.

24. The method of claim 23, wherein the agent is selected from the group consisting of L-lysine, L-valine, D-alanine, D-tyrosine, L-arginine, an analog or structural derivative thereof, and combinations thereof.

25. The method of claim 23, wherein the agent is L-lysine or an analog or structural derivative thereof.

26. A method of treating irritable bowel syndrome (IBS) with diarrhea, the method comprising administering an effective amount of an agent that inhibits the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile and/or Gly-Gly-Phe;
and combinations thereof.

27. The method of claim 26, wherein the agent is selected from the group consisting of L-lysine, L-valine, D-alanine, D-tyrosine, L-arginine, an analog or structural derivative thereof, and combinations thereof.

28. The method of claim 26, wherein the agent is L-lysine or an analog or structural derivative thereof.

29. A method of treating pain in a person infected with human immunodeficiency virus (HIV), the method comprising administering an effective amount of an agent that inhibits the transmembrane transport of an endogenous opioid peptide by the endogenous opioid peptide transport system;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine, stimulation of the transport of endogenous opioid peptide by the tripeptides Gly-Gly-Ile and/or Gly-Gly-Phe;
and combinations thereof.

30. The method of claim 29, wherein the need to administer a pain relieving narcotic is reduced.

31. The method of claim 29, wherein the person infected with HIV has a history of drug abuse.

32. The method of claim 29, wherein person with HIV has opiate-resistant pain.

33. The method of claim 29, wherein the agent is selected from the group consisting of L-lysine, L-valine, D-alanine, D-tyrosine, L-arginine, an analog or structural derivative thereof, and combinations thereof.

34. The method of claim 29, wherein the agent is L-lysine or an analog or structural derivative thereof.

35. An isolated polynucleotide, said polynucleotide hybridizing under standard hybridization conditions to a polynucleotide sequence that encodes an endogenous opioid peptide transport system polypeptide;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile or Gly-Gly-Phe;
and combinations thereof.

36. An isolated polynucleotide encoding an endogenous opioid peptide transport system polypeptide;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile or Gly-Gly-Phe;
and combinations thereof.

37. A plasmid comprising the isolated polynucleotide of claim 35.

38. The plasmid of claim 37, wherein the plasmid comprises an expression vector.

39. An isolated host cell comprising the isolated polynucleotide of claim 35.

40. The isolated host cell of claim 39 demonstrating transient expression of the encoded endogenous opioid peptide transport system polypeptide.

41. The isolated host cell of claim 39 demonstrating stable expression of the encoded endogenous opioid peptide transport system polypeptide.

42. An isolated polypeptide having at least 70% sequence identity with an endogenous opioid peptide transport system polypeptide;
wherein the endogenous opioid peptide transport system exhibits at least one functional activity selected from the group consisting of:
upregulation of the transport of endogenous opioid peptide by the Tat protein encoded by the human immunodeficiency virus type I;
transport of an endogenous opioid peptide is coupled to a sodium gradient;
transport of an endogenous opioid peptide is coupled to a chloride gradient;
inhibition of the transport of endogenous opioid peptide by L-lysine;
stimulation of the transport of endogenous opioid peptide by the tripeptides Gly-Gly-Ile and/or Gly-Gly-Phe;
and combinations thereof.

43. A transgenic non-human animal comprising the isolated polynucleotide of claim 35.

44. A non-human animal having a knockout mutation in one or more alleles encoding a polypeptide of claims 42.
